(19) United States
(12) Patent Application Publication
(10) Pub. No.: US 2014/0147464 A1
(43) Pub. Date: May 29, 2014

(54) METHOD AND COMPOSITION FOR INDUCING AUTOPHAGY

(71) Applicant: NATIONAL YANG-MING UNIVERSITY, Taipei (TW)

(72) Inventors: Rong-Tsun Wu, Taipei (TW); Lin-Yea Horng, Taipei (TW); Hui-Ching Sung, Taipei (TW); Li-Wen Chen, Taipei (TW)

(73) Assignee: NATIONAL YANG-MING UNIVERSITY, Taipei (TW)

(21) Appl. No.: 13/685,292

(22) Filed: Nov. 26, 2012

(51) Int. Cl. A61K 36/074; A61K 31/575

(52) U.S. Cl. CPC A61K 36/074; A61K 31/575
USPC 424/195.15; 514/179

(57) ABSTRACT

A method for inducing autophagy in a subject having an autophagy defect is provided. The method of the present invention includes the step of administering to the subject a therapeutically effective amount of a Ganoderma lucidum extract, wherein the autophagy enhances clearance of protein aggregates in the subject.

### BACKGROUND OF THE INVENTION

1. Field of the Invention

The present invention relates to methods for inducing autophagy, and relates particularly to a method for inducing autophagy in a subject by administering to the subject the extract of Ganoderma lucidum.

2. Description of Related Art

Autophagy or “self digestion process” is an important physiological process that targets cytosolic components such as proteins, protein aggregates and organelles for degradation in lysosomes. The autophagic process is also essential for maintaining neuronal homeostasis, and its dysfunction has been directly linked to an increasing number of diseases.

Autophagy serves the purpose of recycling intracellular nutrients in order to sustain cell metabolism during starvation, and it serves also to eliminate damaged organelles and proteins that have accumulated during times of stress.

Defective autophagy is a major contributor to diseases which may be, but not limited to, neurodegeneration, liver disease, and cancer. A lot of human neurodegenerative diseases are associated with aberrant mutant and/or polyubiquitinated protein accumulation and excessive neuronal cell death.

Nerve growth factor (NGF) was isolated in 1951, and it is the first neurotrophic factor isolated. NGF is produced by astrocytes during development and in the mature animal, and is critical for synaptic plasticity and establishment of functional neuronal circuits. The importance of endogenous NGF for mediating survival and function of basal forebrain cholinergic neurons have been demonstrated, as partial depletion of this trophic factor is associated with measurable deficits in learning and memory.

NGF has shown potential neuroprotective effects in several models. In addition, NGF can protect against neuronal death caused by mitochondrial toxins such as 3-NP and MPTP in the rats. Therefore, NGF was thought to play a pivotal role for pharmacological applications in the treatment of neurodegenerative diseases. However, NGF access is restricted by the blood-brain barrier (BBB) and it is easily metabolised when administered peripherally, thus, it can only be used when directly injected into the brain. When NGF is infused intra-cerebroventricularly, it is also associated with several adverse effects, thus making this delivery route impractical. Therefore, regulating endogenous NGF expression might be a novel therapeutic strategy in neurodegenerative diseases.

Neurodegeneration is a general term for the progressive loss of structure or function of neurons. Many neurodegenerative diseases including Parkinson's disease (PD), Alzheimer's disease (AD), Huntington's disease (HD) and amyotrophic lateral sclerosis (ALS) occur as a result of neurodegenerative processes. Recently, many similarities of neurodegenerative diseases are found, which relate these diseases to one another. For instance, several neurodegenerative diseases are associated with the aggregation of misfolded proteins, which is also known as the atypical protein assemblies.

Huntington's disease (HD) is an autosomal-dominant neurodegenerative disease caused by an abnormal expansion of a CAG trinucleotide repeat in exon 1 of the huntingtin (Htt) gene. The major characteristic of HD is the formation of mutant Htt (mHtt) aggregates of the affected striatal neurons in HD animals and patients. The expansion of this polyglutamine (polyQ) stretch of the Htt gene to more than 37 glutamines or a short N-terminal fragment encoding the polyQ stretch is enough to cause aggregates in mice and in cell models of the disease.

Ganoderma lucidum is one of the most popular medicinal fungi with a long history of use in Asian countries. A great deal of work has been carried out on the therapeutic potential of Ganoderma lucidum. The most important pharmacologically active small molecule constituents of Ganoderma lucidum are triterpenoids, which have been reported to possess hepatoprotective, anti-hypertensive, hypocholesterolemic, anti-histaminic, anti-tumour and anti-angiogenic activities. However, its property of promoting intelligence has not been sufficiently explored.

### SUMMARY OF THE INVENTION

In an aspect of the present invention, a method for inducing autophagy in a subject having an autophagy defect is provided. In accordance with the present invention, the method includes administering to the subject a therapeutically effective amount of a Ganoderma lucidum extract, wherein the autophagy enhances clearance of protein aggregates in the subject.

In one embodiment of the present invention, the autophagy defect is in a cell expressing protein aggregates in the subject, and wherein the cell is a neuronal or glial cell. In one embodiment of the present invention, the protein aggregate is an aggregate selected from the group consisting of hungtingtin, amyloid β (Aβ), α-synuclein, tau, superoxide dismutase 1 (SOD1), variants and mutated forms thereof, and a combination thereof.

In one embodiment of the present invention, the autophagy defect is one disease selected from the group consisting of neurodegenerative disease, Crohn's disease, aging, heart disease and liver disease. In one embodiment of the present invention, the neurodegenerative disease is one selected from the group consisting of Huntington's disease, Alzheimer's disease, Parkinson's disease, amyotrophic lateral sclerosis (ALS) and fatal familial insomnia.

In accordance with the present invention, the Ganoderma lucidum extract is administered orally to the subject.

In another aspect of the present invention, a method for activating nerve growth factor (NGF) in a subject having an autophagy defect is provided, wherein the NGF activates autophagy in the subject, and wherein the autophagy enhances clearance of protein aggregates in the subject. In accordance with the present invention, the method includes administering to the subject a therapeutically effective amount of a Ganoderma lucidum extract.

In one embodiment of the present invention, the autophagy defect is in a cell expressing the protein aggregates in the subject.

In one embodiment of the present invention, the protein aggregate is an aggregate selected from the group consisting of hungtingtin, amyloid β, α-synuclein, tau, superoxide dismutase 1, variants and mutated forms thereof, and a combination thereof.

In one embodiment of the present invention, the protein aggregate is one or more of hungtingtin, amyloid β, α-synuclein, tau, superoxide dismutase 1, variants and mutated forms thereof.

In another aspect of the present invention, a method for preventing memory loss in a subject is provided. In accordance with the present invention, the method includes administering to the subject a therapeutically effective amount of a Ganoderma lucidum extract, wherein the Ganoderma lucidum extract activates autophagy in the subject.

In one embodiment of the present invention, the Ganoderma lucidum extract induces nerve growth factor (NGF) to activate autophagy in the subject. In one embodiment of the present invention, the autophagy enhances protein clearance in the subject.

In accordance with the present invention, the subject has an autophagy defect. In one embodiment of the present invention, the autophagy defect is a neurodegenerative disease selected from the group consisting of Huntington's disease, Alzheimer's disease, Parkinson's disease, amyotrophic lateral sclerosis and fatal familial insomnia.

In another aspect of the present invention, a composition for inducing autophagy in a subject having an autophagy defect is provided, wherein the method includes a ganoderic acid and a pharmaceutical acceptable carrier. In one embodiment of the present invention, the ganoderic acid is one or more selected from the group consisting of Ganoderic acid C2, Ganoderic acid A, Ganoderic acid H, Ganoderenic acid D, Ganoderenic acid D, and 12-acetoxyganoderic acid F.

### BRIEF DESCRIPTION OF THE DRAWINGS

Ganoderma lucidum is abbreviated as GaLu in this specification and drawings.

FIG. 1A to FIG. 1E show the effects of NGF in mHtt74Q-expressing PC12 cell model. (A) FMI ratio of cells treated with NGF. Histograms representing the quantification of FMI measured in two independent experiments (n=6). (B) the shift to the left of the fluorescence distribution, indicating reduced aggregates, in cells expressing mHtt-74Q and treated with NGF 50 ng/ml. The magnitude of green fluorescence is measured on the X-axis while the number of cells exhibiting that degree of fluorescence is depicted on the Y-axis. Data are mean±SD. (**P<0.01 compared with No Dox; ^{#}P<0.05, compared with Dox, Student's t-test.). (C) NGF enhanced the autophagosome formation, which may facilitate the degradation of mHtt. Autophagic vacuoles in cells were stained using MDC. The enhanced MDC stain was shown in NGF-treated cells. A white arrow indicates the co-localization of MDC and EGFP in NGF-treated cells, while the yellow arrows indicate the EGFP aggregates and MDC-stained puncta in the Dox-treated cells. There was rare co-localisation of EGFP and MDC in Dox cells. (Scale bar: 10 μm). (D) Quantification of the MDC stain. Results were expressed as puncta area per cell relative to the untreated control (no dox). (E) Western blot of LC3-I and LC3-II protein levels in each condition is shown (Rapamycin 200 nM as a positive control for induction of autophagy) (n=3). *P<0.05, **P<0.01 compared to No Dox, ^{#}P<0.05, ^{#}P<0.01 compared to Dox, Student's t-test.

FIG. 2A to FIG. 2G show Ganoderma lucidum, an astrocytic NGF inducer, regulated mitochondrial biogenesis in PC12 cells and attenuated mHtt-induced impairments in an HD cell model. Primary astrocytes were treated with GaLu for 6 hours for mRNA analysis (A) or for 24 hours for protein analysis (B). (C) Western blot showing NGF in 20-fold concentrated conditioned media collected from astrocyte cultures treated with GaLu (GaLu-ACM) for 24 hours. (D) Inhibition of mHtt-74Q aggregates by GaLu-ACM measured by flow cytometry. Histograms representing the quantification of FMI measured in two independent experiments (n=6). (E) The shift to the left of fluorescence distribution in cells expressing mHtt-74Q and treated with GaLu-ACM 100 μg ml⁻¹, indicating a reduced number of aggregates. (F) GaLu enhanced MDC intensity. The co-localization of MDC and EGFP in GaLu-treated cells is indicated by the white arrow. (Scale bar: 10 μm). Quantification of MDC staining, showed in puncta area per cell relative to untreated control (no dox). (G) Western blot of LC3-I and LC3-II protein levels in each condition is shown (Rapamycin 200 nM was used as positive control for induction of autophagy) (n=3). *P<0.05, **P<0.01 compared to No Dox, ^{#}P<0.05, ^{##}P<0.01 compared to Dox, Student's t-test.

FIG. 3 shows that Ganoderma lucidum specifically increased NGF mRNA expression in primary astrocytes. Astrocytes treated with Ganoderma lucidum for 6 hours and RT-PCR was performed to analyze the expression of neurotrophic factors (NGF, IGF-1, bFGF and BDNF).

FIG. 4A to FIG. 4C 4 show the astrocytic NGF inducer, Ganoderma lucidum extract induced neurite outgrowth in PC12 cells. Conditioned media from primary astrocytes treated with/without Ganoderma lucidum (Ganoderma lucidum-ACM) for 24 hours were collected. PC12 cells were treated with GaLu-ACM or NGF.

FIG. 5A to FIG. 5J 5 show the identification of constituents from Ganoderma lucidum and the effects of ganoderic acid C₂ in PC12 cells and an HD model. (A) HPLC analysis of GaLu ethanol extracts. Reverse-phase HPLC profile, column: Nucleosil C18 (4.6 mm×250 mm; 5 μm). The mobile phase consisted of 0.1% aqueous acetic acid and acetonitrile using a linear gradient program of 30-32% acetonitrile in 0-40 mins, 32-40% acetonitrile in 40-60 mins, 40% acetonitrile in 60-65 mins, 40-82% acetonitrile in 65-70 mins; flow rate: 0.8 ml/min; detection wavelength: 254 nm. A total of 6 triterpenoids were speculated. (B) Chemical structures of the 6 triterpenoids identified from the GaLu extracts (A-F). (C) Real time PCR analysis of NGF mRNA expression in astrocytes treated with each constituent for 6 h. The expression of p-actin was used as the internal control. (D) Potency was tracked by determining the concentration of the compound required to increase activity by 50% (EC1.5), and the maximum activation potential was listed. (E) PC12 cells were treated with GaLu-ACM or NGF (positive control) for 24 h, and the neurite outgrowth activity was revealed by the percent of neurite outgrowth. (F) Potency was tracked by determining the concentration of the compound required to increase the activity by 50% (EC1.5), and the maximum activation potential is listed. Results are expressed as the relative index of control±SD of at least three independent measurements. (*P<0.05, **P<0.01, one-way ANOVA followed by Tukey's multiple comparison test). (G) Inhibition of mHtt-74Q aggregates by ganoderic acid C₂-ACM measured by flow cytometry. FMI ratio of cells treated with ganoderic acid C₂-ACM. Histograms representing the quantification of FMI measured in two independent experiments (n=6). (H) The shift to the left of the fluorescence distribution in cells expressing mHtt-74Q and treated with ganoderic acid C₂-ACM 20 μg/ml, indicating a reduction in aggregates. (I) Ganoderic acid C₂-ACM enhanced MDC intensity. The white arrow indicates the co-localization of MDC and EGFP in ganoderic acid C₂-ACM-treated cells (Scale bar: 10 μm). Quantification of MDC staining is shown for puncta area per cell relative to untreated control (no dox). (J) Western blot of LC3-I and LC3-II protein levels in each condition is shown (Rapamycin 200 nM was used as positive control for induction of autophagy) (n=3). *P<0.05, **P<0.01 compared to No Dox, ^{#}P<0.05, ^{##}P<0.01 compared to Dox, Student's t-test.

FIG. 6A to FIG. 6G show Ganoderma lucidum extract treatment improved behavioral performance in the 3-NP model.

FIG. 7A and FIG. 7B show brief sleep deprivation in C57BL/6J mice impaired LTP. At first, mice were fed with Ganoderma lucidum (20, 50, 125 mg/kg/day) for 3 days. On the fourth day, mice were deprived of sleep for 5 h by gentle handling. After sleep deprivation, half of the mice were sacrificed for electrophysiological experiment, and the mice were left back into the cage. 24 h later, the mice were sacrificed for electrophysiological experiment of rebound test (A). The maintenance of TBS induced LTP was significantly disrupted in slices from sleep-deprived (SD) mice (B) (0=0.002).

FIG. 8A and FIG. 8B show the effect of Ganoderma lucidum fed mice on the field excitatory post-synaptic potentials (fEPSP) of the hippocampus CA1 monitoring by MED64 system. 4 to 6 week-old C57BL/6J mice were fed with Ganoderma lucidum as described. (A) After 5 h sleep deprivation, long-term potentiation (LTP) was induced in different groups (control, sleep deprivation (SD), Ganoderma lucidum fed 20 mg/kg/day). (B) After 24 h rebound to sleep, mice were sacrificed for fEPSP record. And LTP was induced in different groups (control, n=6; sleep deprivation (SD), n=7, Ganoderma lucidum fed 20 mg/kg/day, n=5).

FIG. 9A and FIG. 9B show the effect of Ganoderma lucidum fed mice on the fEPSP of the hippocampus CA1 monitoring by MED64 system. 4-6 week-old-C57BL/6J mice were fed with Ganoderma lucidum as described. (A) After 5 h sleep deprivation, LTP was induced in the different groups (control, sleep deprivation (SD), Ganoderma lucidum fed 50 mg/kg/day). (B) After 24 h rebound to sleep, mice were sacrificed for fEPSP record. And LTP was induced in different groups (control, n=6; sleep deprivation (SD), n=7; Ganoderma lucidum fed 50 mg/kg/day, n=5).

FIG. 10A and FIG. 10B show the effect of Ganoderma lucidum fed mice on the fEPSP of the hippocampus CA1 monitoring by MED64 system. 4-6 week-old-C57BL/6J mice were fed with Ganoderma lucidum as described. (A) After 5 h sleep deprivation, LTP was induced in different groups (control, sleep deprivation (SD), Ganoderma lucidum fed 125 mg/kg/day). (B) After 24 h rebound to sleep, mice were sacrificed for fEPSP record. And LTP was induced in different groups (control, n=6; sleep deprivation (SD), n=7; Ganoderma lucidum fed 125 mg/kg/day, n=5-7).

FIG. 11 shows sleep deprivation and passive avoidance task in C57BL/6J mice. At first, C57BL/6J mice were subjected to passive avoidance training. After the behavioural session, control animals were returned to their home cages and the remaining mice were deprived of total sleep for 5 h. Immediately after the period of sleep deprivation, the first session was performed. After this session, all animals were kept in their home cages until the second test session.

FIG. 12A to FIG. 12D show body weight and food intake in sleep deprived mice fed with three doses of Ganoderma lucidum extract. 8-week old C57BL/6J mice were firstly fed with three doses (20, 50 and 125 mg/kg/day) of Ganoderma lucidum. Control mice were fed with chow. (A) Mice were weighed before being fed with Ganoderma lucidum (day 1), Ganoderma lucidum-fed duration (day 1-5) and sleep deprivation. (B) Food intake measured during Ganoderma lucidum-fed period. Results were shown as mean value of body weight and food intake per mouse between five groups. (C) The training session of different groups after Ganoderma lucidum treatment for 3 days. (D) The average times mice had been shocked in different groups. SD groups (n=11), Control and Ganoderma lucidum-fed groups (n=9).

FIG. 13 shows the effects of Ganoderma lucidum on sleep deprived C57BL/6J mice in the passive avoidance task. Latency(s) to enter the dark chamber of a passive avoidance apparatus in the test sessions (means±SE) presented by mice that were sleep deprived for 5 h or kept in their home cages (control) after training session. Data of 24 h rebound to sleep are also presented. #p<0.001 compared to control group (Student's t test). *p<0.05, **p<0.01, ***p<0.001 compared to SD control. Control (n=12), SD (n=15), each Ganoderma lucidum treated group (n=12).

FIG. 14A to FIG. 14C show that autophagy increased in Ganoderma lucidum fed mice after 5 h sleep deprivation. 8-week old mice were fed with Ganoderma lucidum of different concentrations (20, 50, 125 mg/kg) for 4 days. After sleep deprivation for 5 h, mice were sacrificed immediately. The hippocampus and cortex were rapidly removed and lysed. The total lysates were assayed for LC-3 expression and cleavage. (A) Lysates in hippocampus. (B) Lysates in cortex. (C) Graphs show statistical results in relative density of bands on the blots estimated by ImageQuant software. Relative densities of the proteins of LC3 to GAPDH are shown. Values are means±SEM (n=6-8), **p<0.01 representing statistical significance was reached compared to control; and #p<0.05, ##p<0.01 represents statistical significance was reached compared to sleep-deprived control.

FIG. 15A to FIG. 15C show that autophagy increased in Ganoderma lucidum fed mice after rebounded to sleep for 24 h. 8 week-old C57BL/6J mice were fed with Ganoderma lucidum of different concentrations (20, 50, 125 mg/kg) for 5 days. After rebound to sleep for 24 h, mice were sacrificed immediately. The hippocampus and cortex were rapidly removed and lysed. The total lysates were assayed for LC-3 expression and cleavage. (A) Lysates in hippocampus. (B) Lysates in cortex. (C) Graphs show statistical results in relative density of bands on the blots estimated by Image Quant software. Relative densities of the proteins of LC3 to GAPDH are shown. Values are means t SEM (n=6-8), **p<0.01 representing statistical significance was reached compared to control; and #p<0.05, ##p<0.01 represents statistical significance was reached compared to sleep-deprived control.

### DETAILED DESCRIPTION OF THE PREFERRED EMBODIMENTS

Various specific details are provided herein to provide a more thorough understanding of the invention.

Ganoderma lucidum Sample Preparation

Dried Ganoderma lucidum (Leyss.ex Fr.) Karst, was soaked in 85% (v/v) ethanol to extract small molecules (MW<1000 daltons) fraction. The extract was concentrated in a rotary vacuum evaporator, lyophilized and stored at −20° C. before use. Separated Ganoderma lucidum small molecular extracts were obtained by HPLC. Reverse-phase HPLC analysis was performed with a Neulcosil C18 column (250 mm×4.6 mm i.d. 5 μm) at room temperature. The mobile phase consisted of 0.1% aqueous acetic acid (v/v, A) and acetonitrile (B) using a linear gradient program of 30-32% B in 0-40 mins, 32-40% B in 40-60 mins, 40% B in 60-65 mins, 40-82% B in 65-70 mins, 82-100% B in 70-85 mins. The effluent was monitored at 254 nm, and a constant flow rate was set at 0.8 ml/min. A Bruker Daltonics ion trap mass spectrometer (Bruker, Billerica, USA) was connected to an Agilent 1100 HPLC instrument via an ESI interface. The LC effluent was introduced into the ESI source in a post-column splitting ratio of 2:1. Ultrahigh-purity helium (He) was used as the collision gas and high-purity nitrogen (N₂) as the nebulizing gas. The optimized parameters in the negative ion mode were as follows: nebulizer, 30 psi; dry gas, 8 L/min; dry temperature, 350° C. For full scan MS analysis, the spectra were recorded in the range of m/z 50 to 1500. A data-dependent acquisition was set so that the two most abundant ions in the full scan MS would trigger tandem mass spectrometry (MSⁿ, n=2).

### Cell Cultures

Astrocyte-enriched cultures were prepared from post-natal one day-old C57BL/6J mice obtained from the Animal Center at the National Yang Ming University, Taiwan. Briefly, cortical tissue was digested with trypsin. The resultant dissociated cells were suspended in DMEM containing 10% FBS and incubated in 100-mm culture dishes. After 3 days in culture, cells were re-fed with fresh 10% FBS/DMEM and maintained at 37° C. for an additional 3 days. The cells were dissociated with trypsin, suspended in 10% FBS/DMEM and incubated in a 10-cm dish for 7-8 days prior to use. Astrocytes prepared by this method consisted of approximately 90-95% astrocytes as determined by immunochemical staining with an antibody against glial fibrillary acidic protein (GFAP), a specific marker for astrocytes. Neural stem cell cultures were prepared from 1 day-old C57BL/6J mice. Cells obtained by trypsinization of cortical tissue were suspended in 100 ml DMEM/F12 medium (2×10⁵ cells/ml) containing 1% N₂, 20 ng/ml EGF, 20 ng/ml bFGF and 100 g/ml BSA in 1 L roller bottles for 7 days for the formation of neurospheres. The neurospheres were examined with an antibody against nestin, a specific marker for neural stem cells. For neuron-glia mixed cultures, neural stem cells were cultured in DMEM/F12 medium containing 1% N₂ and 10% FCS for differentiation for 7 days until matured. PC12 cells were maintained in DMEM supplemented with 10% heat-inactivated horse serum and 5% FBS. All cultures were maintained at 37° C. in a humidified atmosphere of 5% CO₂/95% air.

mHtt74Q-Expressed PC12 Cell

Mammalian expression vectors comprising EGFP (pEGFP-C1, Clontech) fused at its C terminus with an HD gene exon 1 fragment with 74 polyglutamine repeats (mHtt-74Q) was a gift from Dr. David C. Rubinsztein's laboratory. The PC12 stable cells were maintained in 100 g/ml hygromycin in standard medium consisting of DMEM with 100 U/ml penicillin/streptomycin, 2 mM L-glutamine, 10% heat-inactivated horse serum, 5% FBS and 200 ug/ml G418 at 37° C., 5% CO₂. The cells were seeded at 2×10⁵ per well in 12-well plates and were induced expression of mHtt-74Q with 200 ng/ml doxycycline for 24 h. The expression of transgenes was switched off by removing doxycycline from the medium. Cells were either left untreated or treated with NGF (10, 50, 100 ng/ml), Ganoderma lucidum-ACM (20, 100, 500 μg/ml) and Ganoderic acid C₂-ACM (4, 20, 100 μg/ml) at the concentrations specified above for 24 h for gene and protein expression analysis, or the cells were left for 48 h for mitochondrial activity and mHtt-74Q aggregation analysis. The cells were then washed twice with 1×PBS and centrifuged. They were either fixed with 1% paraformaldehyde for 20 mins for FACS analysis (BD Biosciences) of EGFP fluorescence of mHtt-74Q followed by analysis of mean fluorescence intensity of 10,000 events by Cellquest software (BD Biosciences) or processed for real time PCR analysis. Monodansylcadaverine (MDC) (Sigma), a fluorescent compound, was used as a tracer for autophagic vacuoles. Cells were stained with 0.05 mM MDC at 37° C. for 30 mins. After incubation, the cells were washed four times with PBS. The samples were mounted and analyzed using a fluorescence microscope (Olympus IX-70 and POT2, USA) with excitation wave of 335 nm and emission of 525 nm. The intensity of the color staining reflected the level of autophagic activity, which was measured with an Image-Pro Plus Analysis system (Media Cybernetics, Bethesda, USA). To quantify MDC punctae, at least 4 random fields were imaged and the average number of punctae area per cell was calculated.

### RNA Isolation and Real Time PCR

RNA was prepared using RNA-Bee™ RNA isolation reagent (Tel-test, Friendswood, Tex.). An aliquot of 5 μg total RNA was incubated with AMV-RT (Promega) to produce the cDNA for RT-PCR analysis of the expression levels of p-actin, NGF and PGC-1α using the ABI Prism 7700 Sequence Detection System and the SYBR Green Master Mix kit (Applied Biosystems, Foster City, Calif.). The expression level of mouse β-actin was used as an internal reference. Relative gene expression levels were calculated with the 2⁻ΔΔCT method. 100-250-bp fragments were amplified using specific primers for each gene (Table 1).

TABLE 1
Primer sequences used in this paper

| | | |
|---|---|---|
| β-actin | 5′ | GACTACCTCATGAAGATCCT |
| | 3′ | CCACATCTGCTGGAAGGIGG |
| NGF | 5′ | TGTCAAGGG AATGCTGAAGT TTAGT |
| | 3′ | AGCGTAATG TCCATGTTGTTCTAC |
| PGC-1α | 5′ | AGCCGTGACCACTGACAACGAG |
| | 3′ | GCTGCATGGTTCTGAGTGCTAAG |
| nucDNA | 5′ | GCCAGCCTCTCCTGATTTTAGTGT |
| (hexokinase 2 gene intron 9) | 3′ | GGGAACACACAAAAGACCTCTTCTGG |
| m [symbol] DNA (16S rRNA) | 5′ | CCGCAAGGGAAAGATGAAAGAC |
| | 3′ | TCGTTTGGTTTCGGGGTTTC |

[symbol]

indicates data missing or illegible when filed

### Western Blot

Cell lysates were prepared using a radioimmunoprecipitation assay lysis buffer and approximately 20 μg of proteins were loaded and western blot analysis was performed. Astrocyte-conditioned medium (ACM) was collected after a 24 h of Ganoderma lucidum treatment and centrifuged at 200×g for 20 mins to remove cell debris. The supernatant was concentrated 20 fold in a lyophilizer before loading. Primary antibodies included a 1:1,000 diluted polyclonal rabbit antibody against the mouse NGF peptide (amino acids 40-63) (Cat no. ab6198, Abcam, Cambridge, UK), a 1:1,000 diluted polyclonal rabbit antibody against the mouse LC3 (Cat no. PM-036, MBL, JAPAN) and a 1:10,000 diluted antibody against GAPDH (Cat no. ab9385, Abcam, Cambridge, UK) that was used as a loading control. Antibody-bound proteins were stained using a horseradish peroxidase-conjugated anti IgG secondary antibody system for enhanced chemiluminescence detection (Amersham, Buckinghamshire, UK).

### Detection of NGF-Like Proteins in Astrocyte-Conditioned Medium (ACM) Using a PC12 Bioassay

To evaluate neurite outgrowth, PC12 cells were plated at a low density (2×10⁴ cells per cm²) onto poly-D lysine-coated 24-well plates. After 24 h, the adherent PC12 cells were washed with PBS and incubated with conditioned medium derived from either untreated or Ganoderma lucidum-treated astrocyte cultures to monitor neurite outgrowth (using a low serum condition in DMEM containing 1% FBS). Eight to ten images per group were photographed under light microscopy, and the percentage of cells with neurites that exceeded (the percentage of cells with neurites exceeding) the diameter of cell body was analyzed by examining 100-200 cells per image. Images were analyzed by an operator blinded to the experimental conditions. For the preparation of ACM, astrocytes were treated with Ganoderma lucidum for 24 hours and were washed with PBS and fed with low serum medium (without Ganoderma lucidum). After 24 hours, the medium was centrifuged at 200×g for 20 mins to remove cell debris, and the supernatant was collected as the ACM and used immediately. Murine NGF 100 ng/ml (Promega Biotech Co., Ltd, USA) was used as positive control.

### Succinate Dehydrogenase (SDH) Assay, Mitotracker Assay and Mitochondrial/Nuclear DNA Ratios

PC12 or mHtt-74Q cells (2×10⁴ cells per well) were plated in 96-well plates. After 24 hours, cells were incubated in Ganoderma lucidum-ACM or NGF-containing media (100 μl per well) for 48 hours. Succinate dehydrogenase activity was normalized to cell protein (measured by BioRed protein kit) and changes in absorbance were measured using a microplate reader (PerkinElmer Life Sciences Wallac Victor2). Activity was expressed relative to the control condition. Mitochondrial content was detected by Mitotracker Green FM staining and mitochondrial membrane potential by Mitotracker Red (tetramethylrhodamine methyl ester (TMRM)) staining (Invitrogen). Cells were incubated with Ganoderma lucidum-ACM or NGF-containing medium for 24 or 48 hours. Cells were washed with serum-free DMEM and stained with 100 nM Mitotracker Green FM or Mitotracker Red (TMRM) for 30 mins. The unstained control samples were incubated with serum-free DMEM containing no dye, but an equivalent concentration of dimethyl sulfoxide (DMSO) was used as the stained sample. After staining, the cells were washed three times with PBS. Stained cells were detected by fluorescence microscopy. For the microplate assay, staining was detected on a fluorescence microplate reader (excitation wavelength of 485 nm, and emission wavelength of 520 nm). Stained (and unstained control) cells were analyzed by flow cytometry (BD Biosciences) followed by analysis of mean fluorescence intensity of 10,000 events by the Cellquest software (BD Biosciences). Mitochondrial/nuclear DNA ratios were analyzed by real time PCR. Cells (2×10⁵ cells per well) were plated in 12-well plates. After 24 hours, the cells were incubated in Ganoderma lucidum-ACM or NGF-containing media for 48 hours. Genomic DNA (containing both mitochondrial and nuclear DNA) was isolated from the cells. DNA (10 ng) was amplified by quantitative real time PCR. Primers were listed in Table 1.

### Animals and 3-NP Intoxication

Sixty 12-week-old C57Bl/6J adult male mice that were obtained from the National Laboratory Animal Center (Taipei, Taiwan) were housed at a constant temperature and supplied with laboratory chow (PMI, Brentwood, Mo., USA) and water ad libitum. The experimental procedure was approved by the Animal Research Committee of National Yang-Ming University, Taiwan. The mitochondria toxin 3-nitropropionic acid (3-NP) (Sigma, France) (Stock 10 mg/ml) was dissolved in 0.1 M phosphate buffered saline (PBS) at a pH of 7.4, and was filtered (Millipore, 0.22 μm) and kept at 4° C. until use. Mice received two daily intraperitoneal (i.p.) injections of the 3-NP solution, 12 hours apart (at 10:00 a.m. and 10:00 p.m. each day) with the following schedule with minor modifications: 3-NP concentrations of up to 600 mg/kg was used to increase the neurodegenerative process: 20 mg/kg×4 injections, 40 mg/kg×4 injections, 60 mg/kg×6 injections (total cumulated dose: 600 mg/kg in 7 days). A total of 60 mice were divided into 5 groups; namely four 3-NP treated groups and a control group of mice that received saline. After 3-NP-induced damage (day 8), mice were then fed with a normal diet or normal diet containing different concentrations of Ganoderma lucidum (24, 60 or 150 mg/kg per day) for 14 days.

### Behavioral Score

Behaviors were graded 0 through to 5 according to the following scale: grade 0, normal behavior; grade 1, general slowness in movement due to mild hindlimb impairment; grade 2, prominent gait abnormality with poor coordination; grade 3, near complete hind-limb paralysis; grade 4, inability to move due to forelimb impairment; and grade 5, recumbency or death. The behaviors of the mice were scored by two independent examiners blinded to the experimental conditions.

### Rotarod Test

Mice in each group were examined for sensorimotor ability using the rotarod test. Before testing, the animals were each trained on the rotarod apparatus for a maximum of 180 s in 3 consecutive sessions for 3 days. Animals that did not master this task were excluded from further experiments. The apparatus consisted of a bar with a diameter of 6.0 cm that was subdivided into four compartments by disks 50-cm in diameter. The bar rotated at an accelerated speed 14 and 22 rpm. For each trial, the duration that the animals were able to stay on the apparatus prior to falling was measured with a maximum trial latency of 180 s. The times of three separate measurements were recorded and averaged.

### Tissue Processing and GFAP Immunohistochemistry

After the completion of behavioral experiments and drug treatments for 2 weeks, all animals were anesthetized with a lethal dose of sodium pentobarbital (i.p.). The mice were perfused with 10 ml of 0.9% NaCl followed by 30 ml of 4% paraformaldehyde in 0.1 M PBS, at pH 7.4. The brains were removed and placed in the same fixative for 24 h. They were then transferred to a 30% sucrose solution in 0.1 M PBS until they sank. The brains were frozen, stored at −70° C. and cut into 30 μm cryostat coronal sections, which were collected free floating for immunohistochemistry. For GFAP immunostaining, frozen sections were prepared as mentioned above and rinsed three times in PBS before blocking with 4% bovine serum albumin. After blocking, sections were incubated overnight at 4° C. in Tris buffer containing the primary monoclonal antibodies that recognize GFAP (1:1000 dilutions, NOVOUS, Littleton, USA), a morphological marker of reactive gliosis. Sections were washed three times with PBS and incubated in 3% H₂O₂ 30 mins for peroxidase blocking. The secondary antibody, rabbit anti-mouse conjugated with horseradish peroxidase (1:200 dilutions; DAKO Kit; Dakocytomation, Glostrup, Denmark) and diaminobenzidine were added, and the sections were analyzed by an examiner blinded to the experimental conditions for gliosis by light microscopy.

### Counting of Nissl Stained Cells

The Nissl technique was used to analyze cellular density around the striatal region after experiment. The previous method was used as described. Briefly, all sections cut throughout the striatum were stained with cresyl violet and observed under a light microscope. Using cresyl violet staining, neurons were identified as the largest cells in the field with typical morphological features which included an abundant cytoplasm, a polygonal shape, and at least one emanating process; while the astroglial cell profiles were distinguished from neurons by their round, small, and hyperchromatic nuclei. Nissl (+)-neurons were counted on the images at every 6 coronal section, and an average of 10 sections per brain was analyzed by examiners blinded to the experimental conditions. One frame for the visual field of 200×200 μm was used for counting and measuring. The packing density (PD) of Nissl (+)-neurons was calculated by using the determined number of cells and the square area of outlined frames in each section analyzed. The following equation was used:

$$PD = \frac{{\sum }_{i = 1}^{n} {N}_{i}}{{\sum }_{i = 1}^{n} {SA}_{i}}$$

wherein PD means packing density (mm⁻²), Nᵢ is the number of counted neurons in i-th section (corrected by Abercrombie's formula), and SAᵢ is the square area of i-th analyzed frame (mm²). The data are the means±S.D. of three animals per group.

### Determination of SDH Activity

The SDH activity in the brain tissue of the control vehicle (3-NP alone or 3-NP plus Ganoderma lucidum-treated groups) was measured as previously described. Approximately 20 sections from each animal were incubated with 0.1 M PBS at 37° C. for 15 mins to activate the SDH. The sections were washed with a large volume of 0.1 M PBS and incubated with 0.3 mM nitroblue tetrazolium, 0.05 M sodium succinate and 0.05 M phosphate buffer (pH 7.6) for 30 mins at 37° C. For the determination of nonspecific staining unrelated to SDH activity, adjacent sections were incubated in the same medium in which succinate was omitted. The sections were rinsed with cold PBS for 5 mins, fixed with 4% paraformaldehyde, rinsed with water and finally dried at room temperature. The intensity of the blue color staining reflected the level of SDH activity, which was measured by an Image-Pro Plus Analysis system (Media Cybernetics, Bethesda, USA). A circular probe was placed on the region of interest to determine the relative optical density (within a range of 0-255 grey levels) of the stain in that part of the tissue. Ten sections per animal (3-4 brains per group) were analyzed by an operator blinded to the experimental conditions.

### Electrophysiological Experiments

C57BL/6J mice of approximately 4 to 6 weeks old were anaesthetized by ether, and the brains of these mice were subsequently obtained and soaked immediately in artificial cerebrospinal fluid (aCSF) containing the following: 122 mM of NaCl, 3.1 KCL, 1.1 mM of MgSO₄.8H₂O, 1.3 mM of CaCl₂.2H₂O, 10 mM of glucose, 0.4 mM of KH₂PO₄, and 25 mM of NaHCO₃. After removal of the cerebellum and the olfactory lobe, one third of the brain was retained, and the vibrating tissue slicer (D.S.K Microslicer, Model DTK-1000) was used to slice the brain into slices of 350 μm. The brain slices were kept in aCSF for 2 hours that were maintained in 95% O₂ and 5% CO₂, allowing the injured parts of the brain to recover. Further, the brain slices, were placed on the MED64 probe (Panasonic; MED-P515AP), and a digital microscope (Olympus, MIC-D) was used to photograph the corresponding positions on the brain slices after suitable adjustments of positions, and a multi-channel recording system (Panasonic, MED64) was used to record electrophysiological responses of the brain slices.

The MED64 (Panasonic) system included probe, connector, integrated amplifier and the Lerformer software 1.5, the center of the probe had an arrangement of 64 microelectrodes. Each microelectrode had size of 50×50 μm², and the distance between each electrode was 150 μm. Before its first use, the probe was soaked in 0.1% polyethylenimine (PEI) in borate buffer (0.15M, pH 4.8) for more than 8 hours to allow coating to occur. Then, the probe was used after being washed with deionized water, and the probe was injected with distilled water, sealed with parafilm, and stored at 4° C.

### Memory Test

The passive avoidance test was used to assess memory behavior associated with the hippocampus of the mice. The mice were placed in between the light and dark compartment, and they generally moved toward the dark room. However, when the mice entered the dark compartment, an electric shock at 0.5 mA was applied continuously for 2 seconds, to make them trained to associate the dark compartment with electric shock. The time latency for the mice to remain in the light compartment was used as a basis to assess the mice's memory, the electric shock in the dark compartment. The time latency of the mice that exceeded 300 seconds in the light compartment was not recorded. The effects of Ganoderma lucidum on the memory of the sleep-deprived mice were observed and compared.

### Sleep Deprivation in Mice

After completion of the passive avoidance test, C57BL/6J mice of approximately 10 weeks old were divided into the sleep deprived group and the control group of mice that stayed in the cage, and all these mice were placed in the same compartment. The sleep-deprived group of mice was subjected to total sleep deprivation for 5 hour. The sleepy state of the mice was then observed subjectively, and when mice were seen to be sleepy, the cage was tapped lightly to prevent them from falling asleep. Sufficient levels of water and food were provided in the cage, and the mice were able to move about freely in the cage while they were awake.

### Statistical Analysis

All results are expressed as mean and ±standard deviation (SD). The significance of differences of the means between more than two groups was determined using a one-way analysis of variance (ANOVA) followed by Tukey's post-hoc test. The Student's t-test was employed for the statistical comparison of paired samples. A P value of <0.05 was considered statistically significant.

NGF Treatment Reduced mHtt-74Q Aggregation in a HD Cell Model.

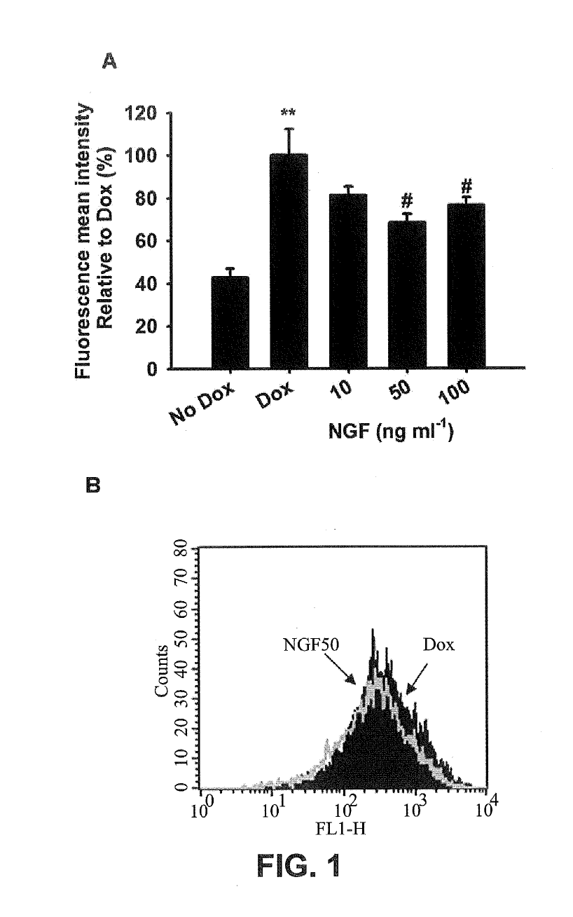

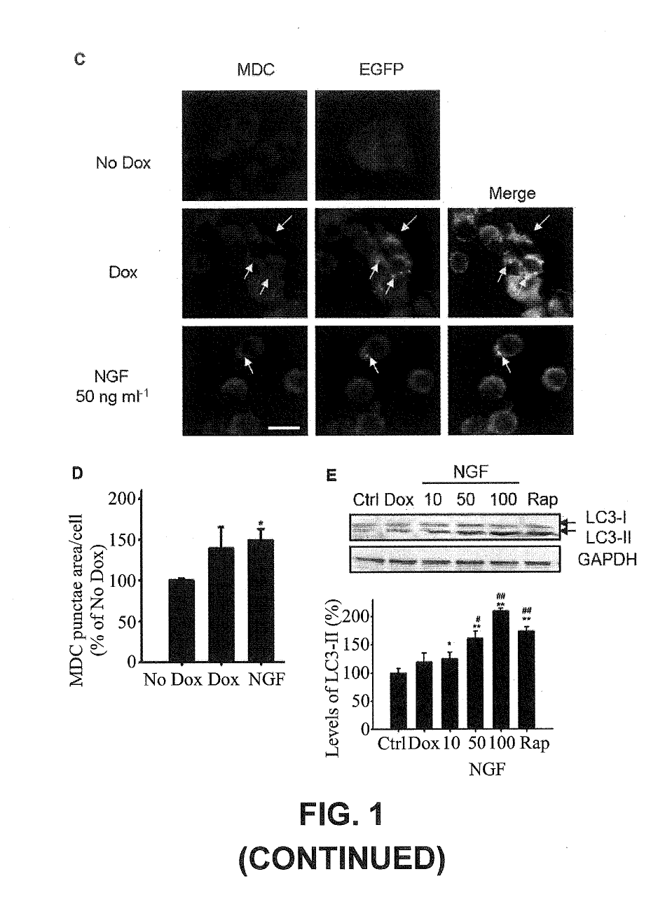

A genetic cell model expressing the Huntington disease (HD) protein (mHtt-74Q) under doxycycline (Dox) control was used to demonstrate the effects of NGF treatment. The content of mHtt-74Q in the HD cell model was identified by FACS analysis. A reduction in mHtt-74Q accumulation was observed upon NGF treatment (FIGS. 1A and 1B). Next, the potential mechanism by which NGF reduces the mHtt aggregates was investigated. To achieve this, the role of autophagy was assessed using MDC staining (FIGS. 1C and 1D) and western blot analysis of LC3-II expression (FIG. 1E), of which LC3-II were specific markers for the autophagolysosome. Autophagic activity was seen to be up-regulated in the NGF-treated cells, and the activity was slightly induced in cells treated with Dox alone. Also, a reduction in enhanced green fluorescent protein (EGFP) aggregates was shown in NGF-treated cells (FIGS. 1A and 1C). The co-localization of EGFP and autophagic vacuoles was further identified in NGF-treated cells, which suggested the involvement, at least in part, of autophagy in the mHtt-74Q clearance. However, the co-localization of MDC-EGFP in dox cells alone was not efficient. Thus, more aggregates were deposited, possibly due to a defect in cargo recognition. Taken together, these results support the view that a more efficient autophagy program triggered by NGF may lead to fewer mHtt aggregates within the cell through more degradation and less aggregate formation.

Ganoderma lucidum Stimulated NGF Expression in Primary Astrocyte Cultures

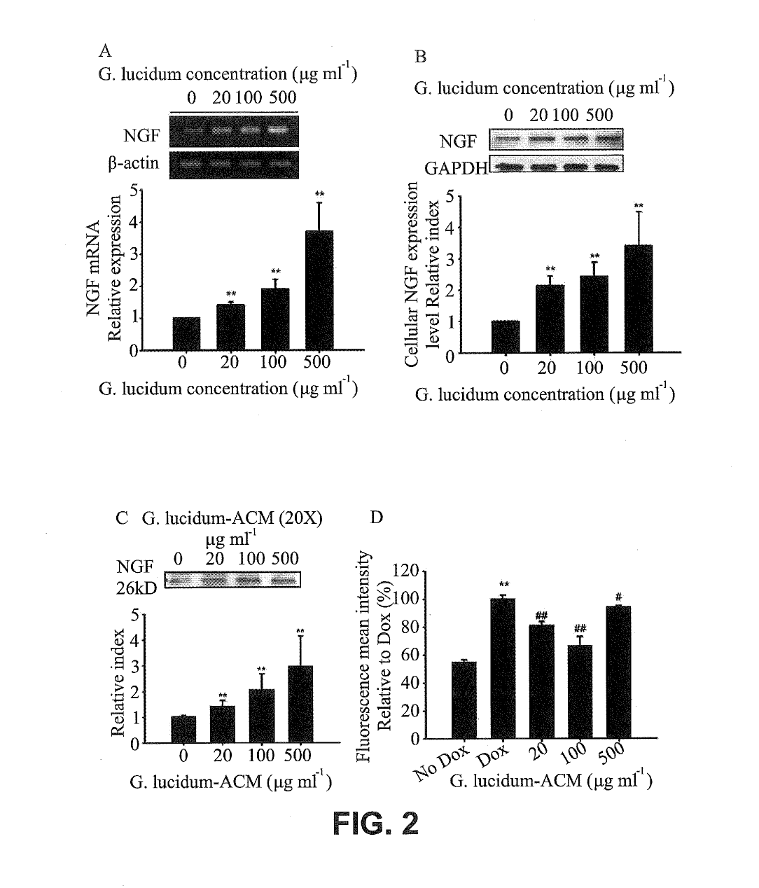

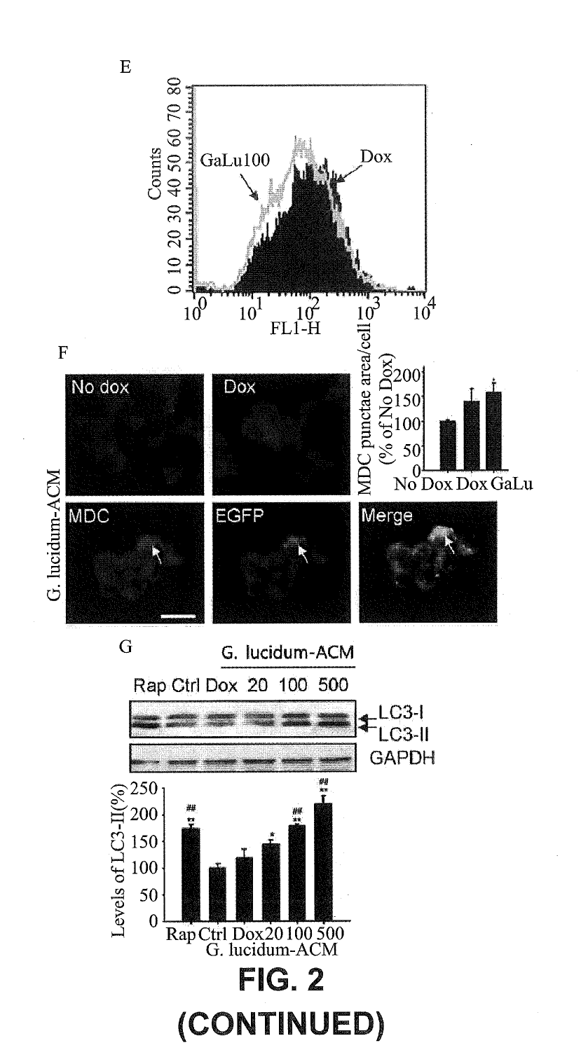

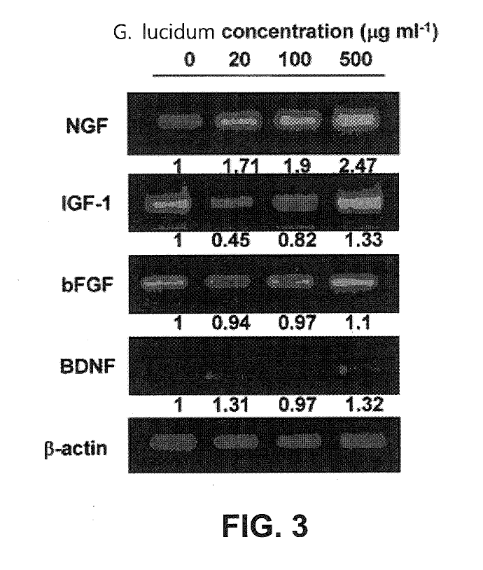

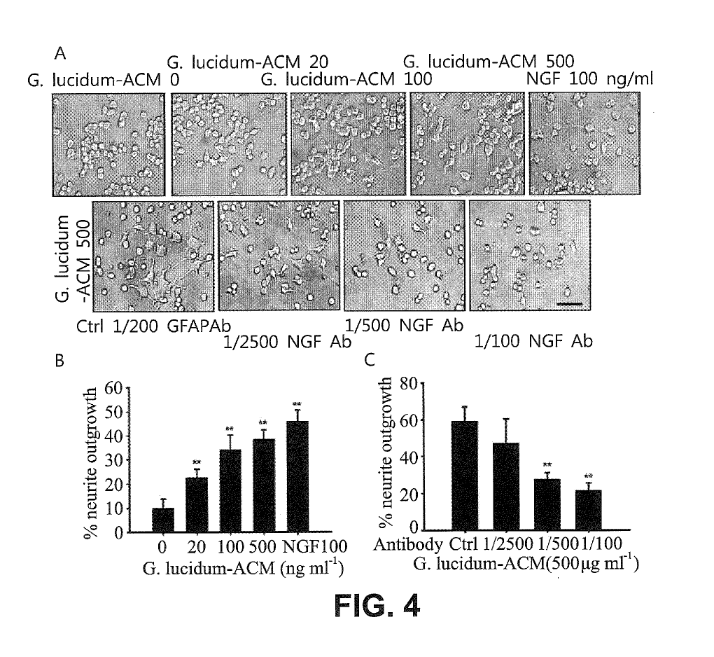

Due to the fact that exogenous NGF cannot cross the blood-brain barrier, thereby limiting its clinical use, the effects of an endogenous astrocytic NGF inducer Ganoderma lucidum was tested. After Ganoderma lucidum treatment, astrocytes demonstrated a dose-dependent increase in NGF mRNA expression, as measured by RT-PCR and real time PCR analysis (FIG. 2A). Ganoderma lucidum treatment also upregulated intracellular NGF protein expression (FIG. 2B). To investigate whether NGF induction was accompanied with an increase in NGF release, NGF levels in 24 h-conditioned media from astrocytes treated with different concentrations of Ganoderma lucidum were analyzed. The results indicated that Ganoderma lucidum treatment enhanced the levels of NGF released into the culture medium in a dose-dependent manner (FIG. 2C). Therefore, Ganoderma lucidum enhanced both the synthesis and secretion of NGF in astrocytes. The specificity of Ganoderma lucidum's effects on NGF expression in primary astrocyte cultures is shown in FIG. 3. When PC12 cells were incubated with the conditioned media from Ganoderma lucidum-treated astrocytes (Ganoderma lucidum-ACM), neurite outgrowth activity was stimulated (FIGS. 4A and 4B). The specific activity of NGF on neurite outgrowth was blocked by co-incubating PC12 cells with 500 μg/ml Ganoderma lucidum-ACM and an NGF-specific antibody (FIGS. 4A and 4C).

Effect of Ganoderma lucidum in an mHtt74Q-Expressing HD Cell Model

The mHtt-74Q content in a Hungtington Disease cell model (cells that express mHtt-74Q) was identified by FACS analysis. Ganoderma lucidum-ACM treatment reduced mHtt-74Q accumulation in cells (FIGS. 2D and 2E). Ganoderma lucidum-ACM treatment also enhanced MDC intensity (FIG. 2F) and LC3-II expression (FIG. 2G).

HPLC and LC-MS Analysis of Ganoderma lucidum: the Identification of Different Ganoderic Acids and their Effects on NGF Stimulation and Neurite Outgrowth Activity

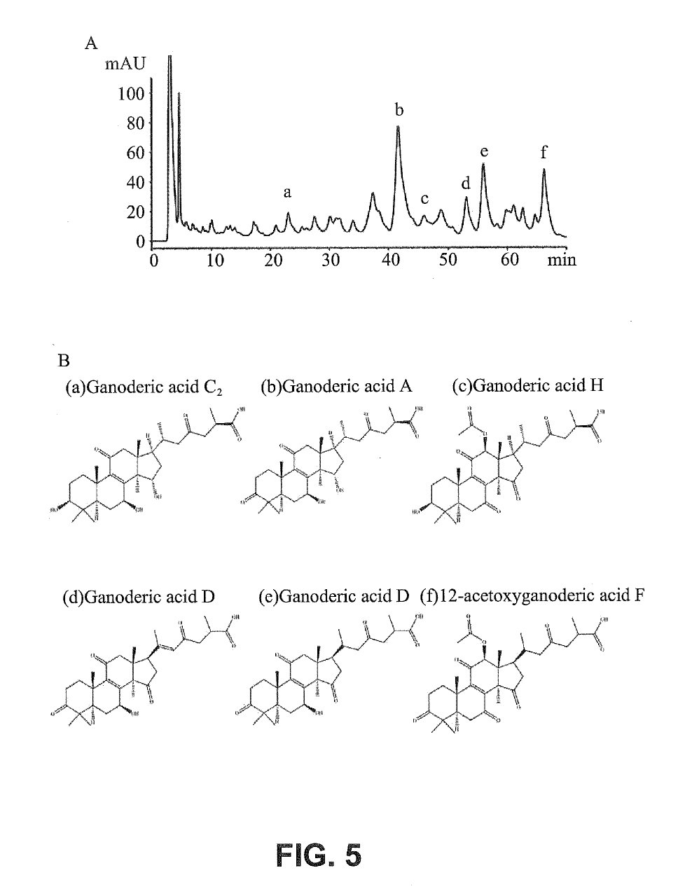

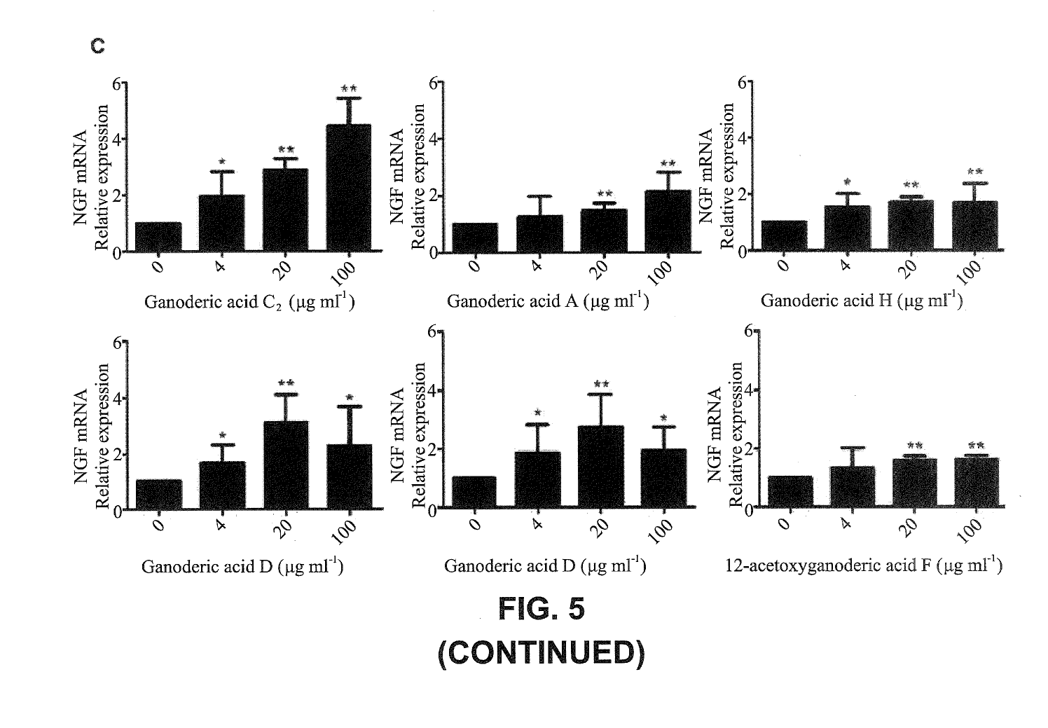

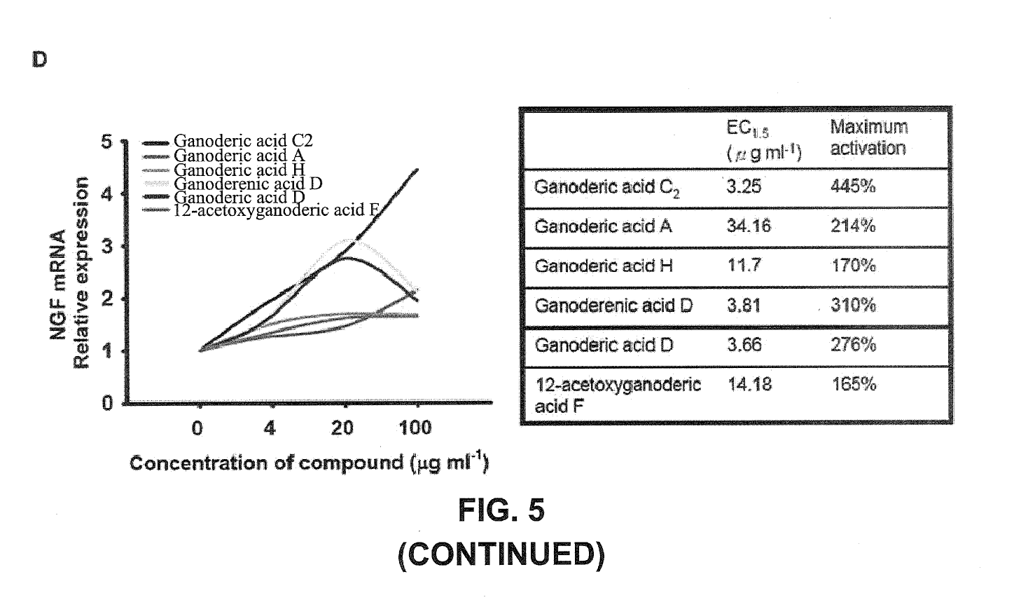

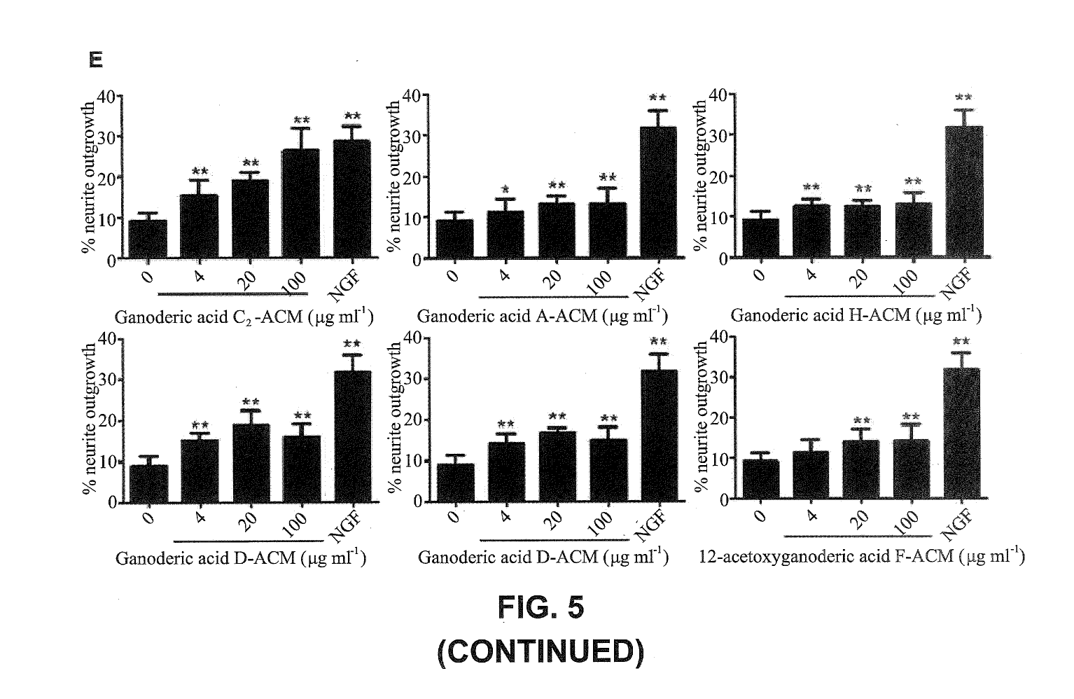

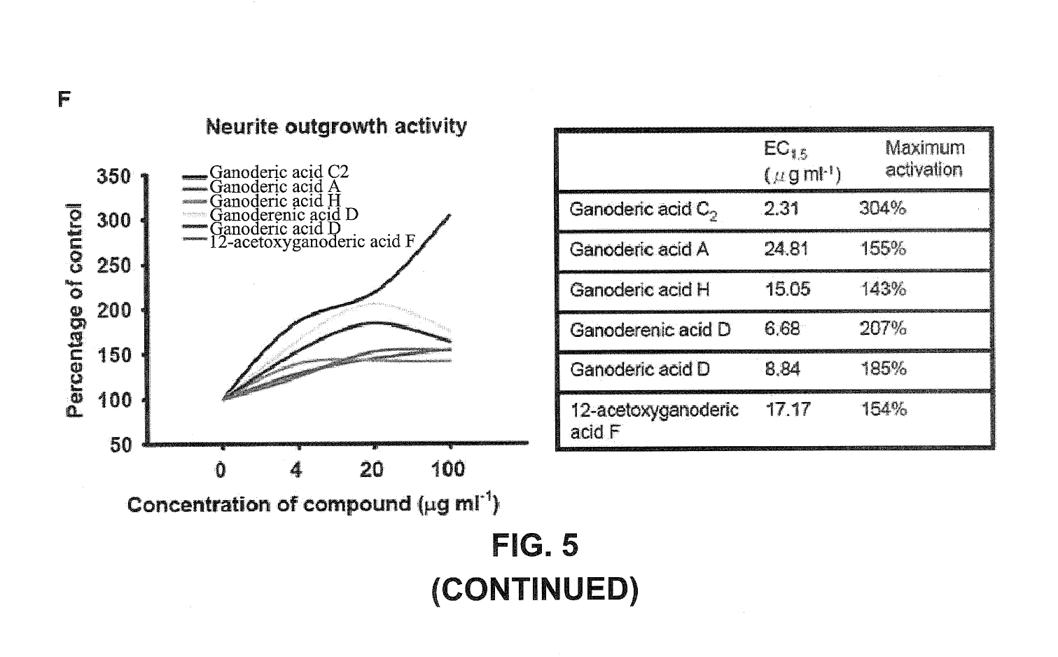

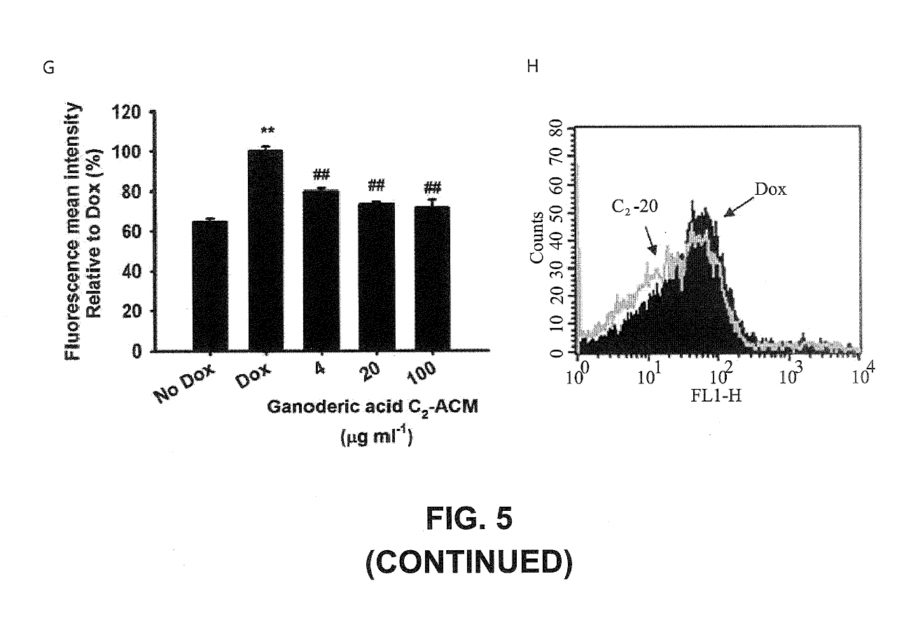

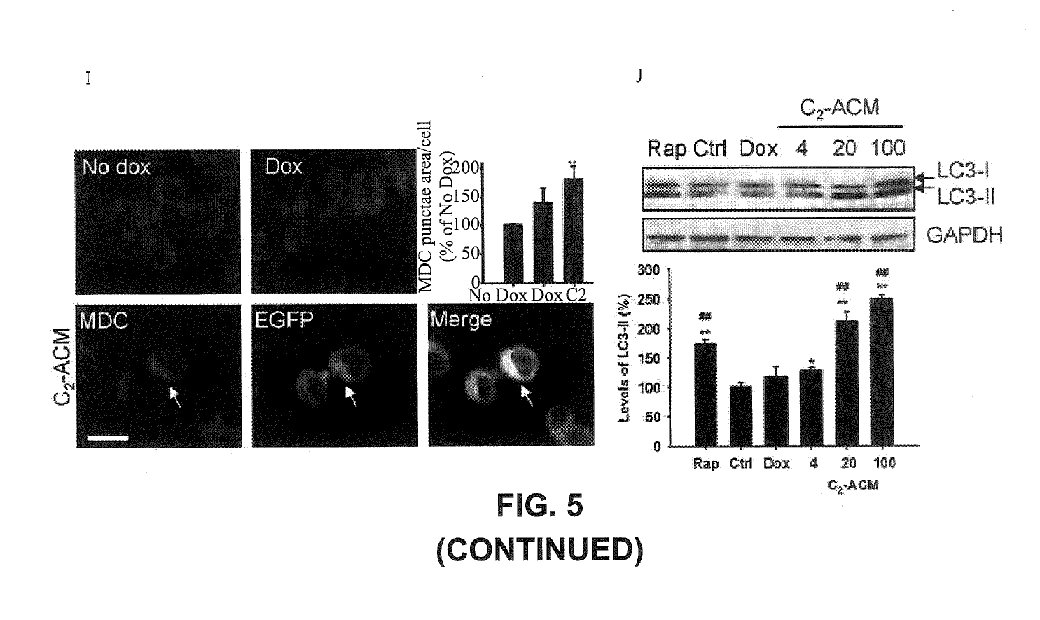

HPLC and LC-MS were performed to identify the active ingredient within the ethanol extract of Ganoderma lucidum. The Ganoderma lucidum fingerprint was obtained by using a reversed-phase HPLC analysis. FIG. 5A shows the HPLC-UV profiles of the ethanol extract of Ganoderma lucidum. To obtain the optimal extraction efficiency and good separation, the extraction and chromatographic conditions were optimized. For the analysis of triterpenoids in the crude extract, a positive/negative ion ESI-MS was used to obtain the molecular mass information. Six fractions were speculated from the ethanol extract of Ganoderma lucidum, and the structures were identified (FIG. 5B). The activities of all of these extracts were tested by means of NGF mRNA induction in astrocytes and a neurite outgrowth assay (FIGS. 5C and 5E). Potency was tracked by determining the concentration of the fraction required to increase activity by 50% (EC1.5) (FIGS. 5D and 5F). NGF stimulation and neurite outgrowth activity were particularly marked in a fraction that was enriched in ganoderic acid C₂.

Effect of Ganoderic Acid C₂ in mHtt74Q-Expressed HD Cell Model

Ganoderic acid C₂-ACM treatment was observed to reduce mHtt-74Q accumulation in the Huntington disease cell model (FIGS. 5G and 5H). By means of MDC staining (FIG. 5I) and western blot analysis of LC3-II expression (FIG. 5J), the upregulation of autophagic activity in ganoderic acid C₂-ACM treated cells were demonstrated.

Effect of Ganoderma lucidum on 3-NP-Induced Mouse Striatal Degeneration

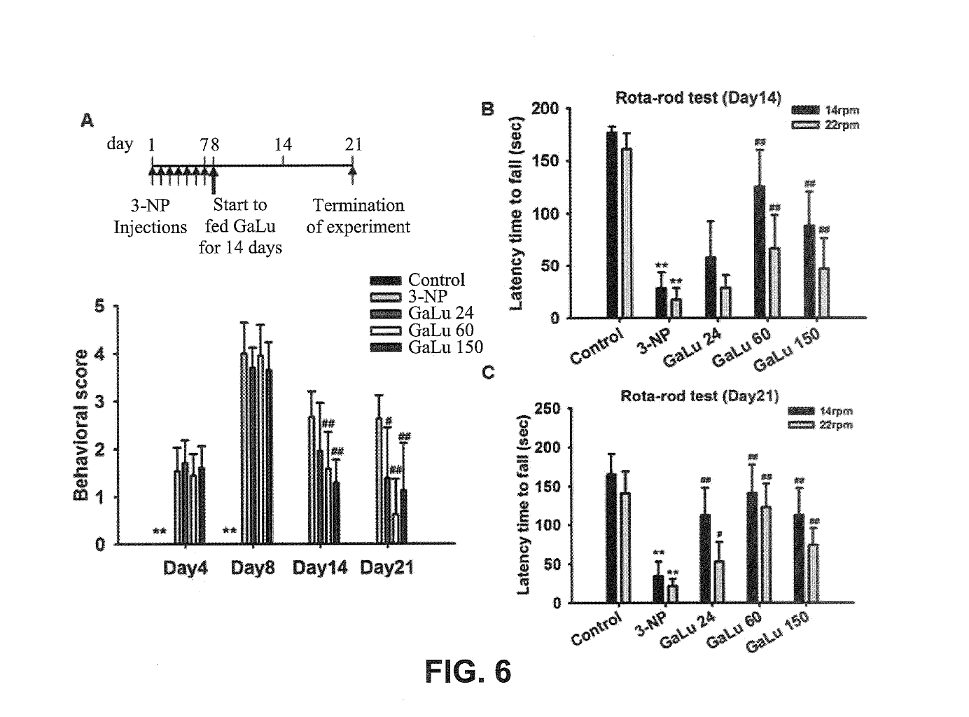

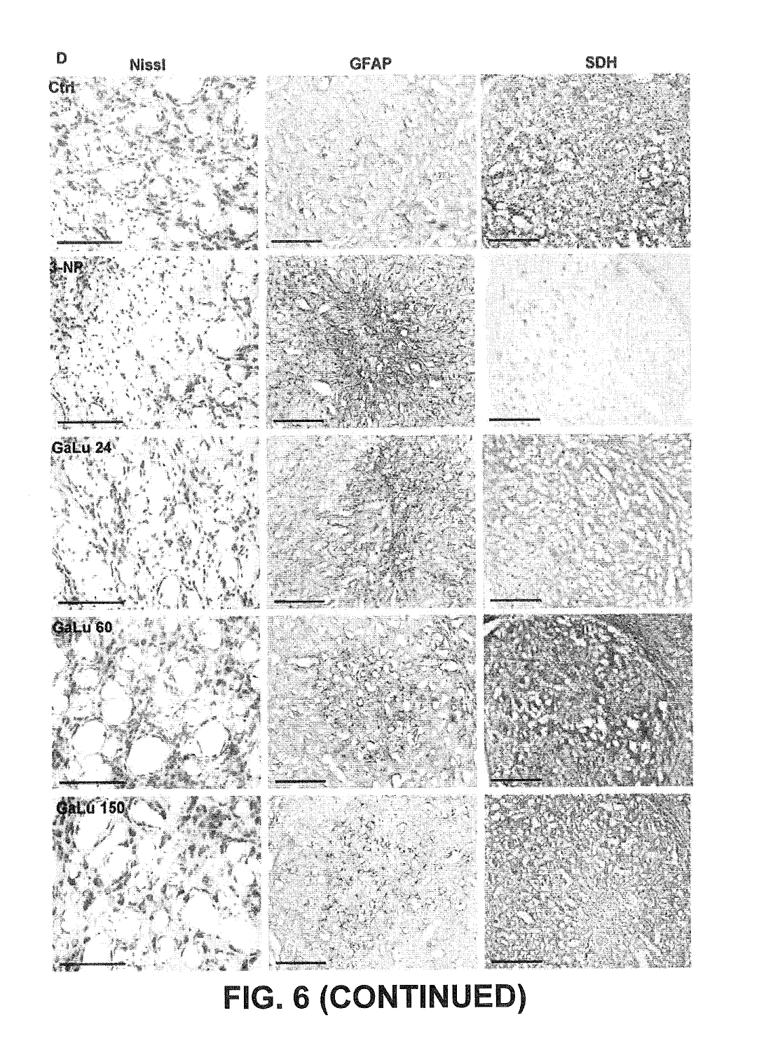

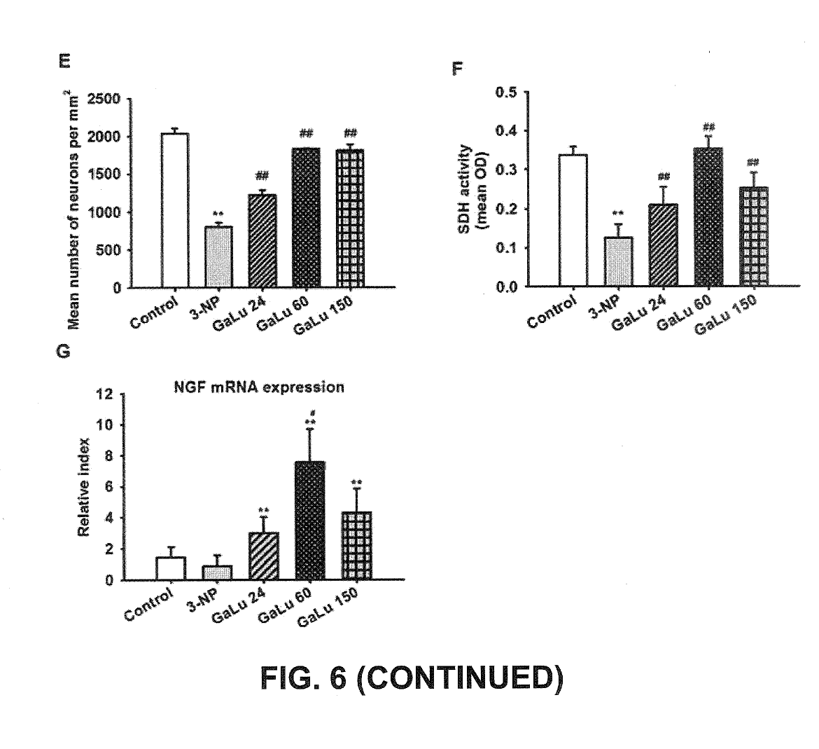

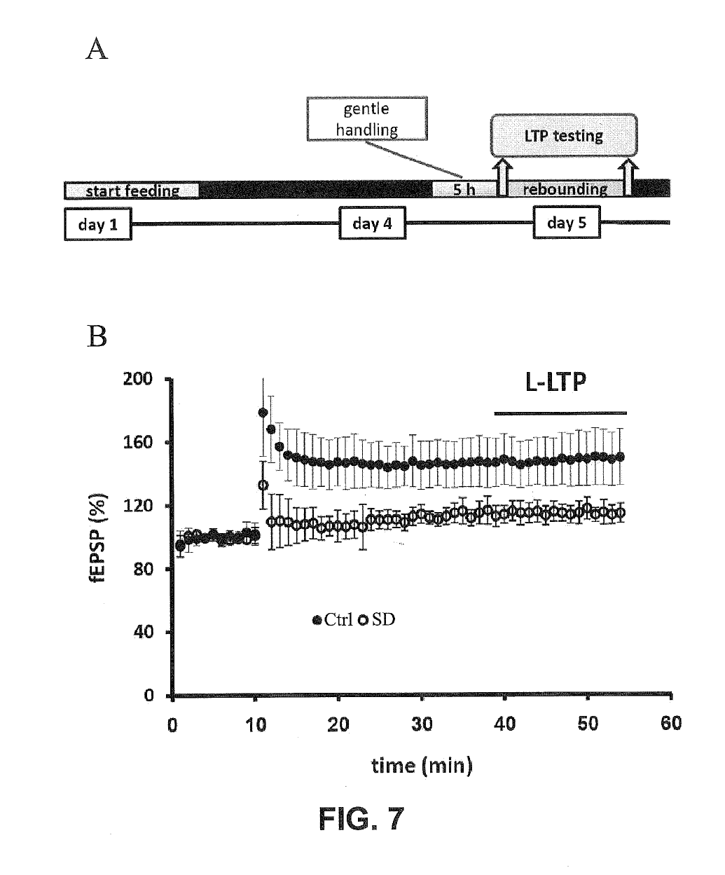

As the neuroprotective effects of NGF in the 3-NP model were observed, this model was used to further evaluate the therapeutic effects of Ganoderma lucidum in vivo. With small modifications of a previous method, 3-NP concentrations of up to 600 mg/kg were used to increase the neurodegenerative progress. After 3-NP-induced damage (day 8), mice were fed different concentrations (24, 60 or 150 mg/kg) of the Ganoderma lucidum diet for 14 days. As shown in FIG. 6A, 3-NP induced severe postural abnormalities on day 8 post-intoxication, and mice on a Ganoderma lucidum diet exhibited an earlier recovery of their behavioural score (60 mg/kg and 150 mg/kg Ganoderma lucidum were fed on day 14, respectively; 24 mg/kg Ganoderma lucidum was fed on day 21). When the Rota-Rod test was performed on day 14 and day 21 to evaluate the recovery of sensorimotor function in the mice, the performance was improved by Ganoderma lucidum treatment at concentrations of 60 and 150 mg/kg on day 14, and 24 mg/kg on day 21 when compared with 3-NP-treated control mice, which remained impaired (FIGS. 6B and 6C). Therefore, treatment with Ganoderma lucidum improved the behavioural deficits compared to the 3-NP-treated control animals. The efficacy of Ganoderma lucidum on reversing the neurotoxicity induced by 3-NP was examined using Nissl, GFAP and SDH staining of coronal brain sections of the mice at the end of the Ganoderma lucidum treatment (FIG. 6D). FIG. 6D shows representative images of mice treated with 3-NP alone and 3-NP followed by the administration of 3 different doses of Ganoderma lucidum. Animals treated with 3-NP alone displayed obvious loss of striatal neurons, but the Ganoderma lucidum-fed groups displayed less neuronal loss in the striata. Moreover, the Ganoderma lucidum diet from 24 to 150 mg/kg showed a dose-dependent effect against 3-NP-induced striatal neuron loss. FIG. 6E represents the quantification of neurons from the Nissl stain, and a significant neuroprotective effect of the Ganoderma lucidum diet was observed. The Ganoderma lucidum diet also attenuated 3-NP-induced GFAP over-activation. There were more GFAP-positive astrocytes around the damaged striatal area in the group that received 3-NP alone than in the 3-NP-Ganoderma lucidum treated groups. 3-NP is a well-known irreversible inhibitor of SDH in vivo. As shown in FIGS. 6D and 6F, treatment with 3-NP induced significant inhibition of SDH activity in the brain of mice treated with 3-NP alone compared to the control. However, the administration of Ganoderma lucidum alleviated the 3-NP-induced inhibition of SDH activity. In conclusion, the results of neuron counting combined with the data from GFAP and SDH activity assays indicated that Ganoderma lucidum provided neuroprotective effects in animals against 3-NP-induced striatal damages. The mRNA isolated from the striata of the 3-NP model with or without Ganoderma lucidum treatments was processed for NGF expression levels. Mice with the Ganoderma lucidum diet had significantly higher NGF expression in the striata (FIG. 6G). This increase was dose-dependent, and Ganoderma lucidum at 60 mg/kg presented the most striking stimulation of NGF expression, a 4.5-fold increase compared to the control and a 7-fold increase compared to the 3-NP group.

Ganoderma lucidum Prevented Sleep Deprivation and Reduces Long Term Potentiation

Mice experiments proved that the memory consolidation of mice can be affected by short term or long term sleep deprivation. It is well known that 5 h sleep deprivation method can affect the long term potentiation (LTP) in the hippocampus of mice. Good performance of LTP was not maintained even after 30 mins of sleep deprivation, and that over time, the LTP of mice decreased, and long term memory was affected.

Mice were fed Ganoderma lucidum for three days, and on the fourth morning, sleep deprivation was (introduced) performed to the mice. After sleep deprivation, the mice were allowed to sleep freely for 24 hours, and then the experiments were performed.

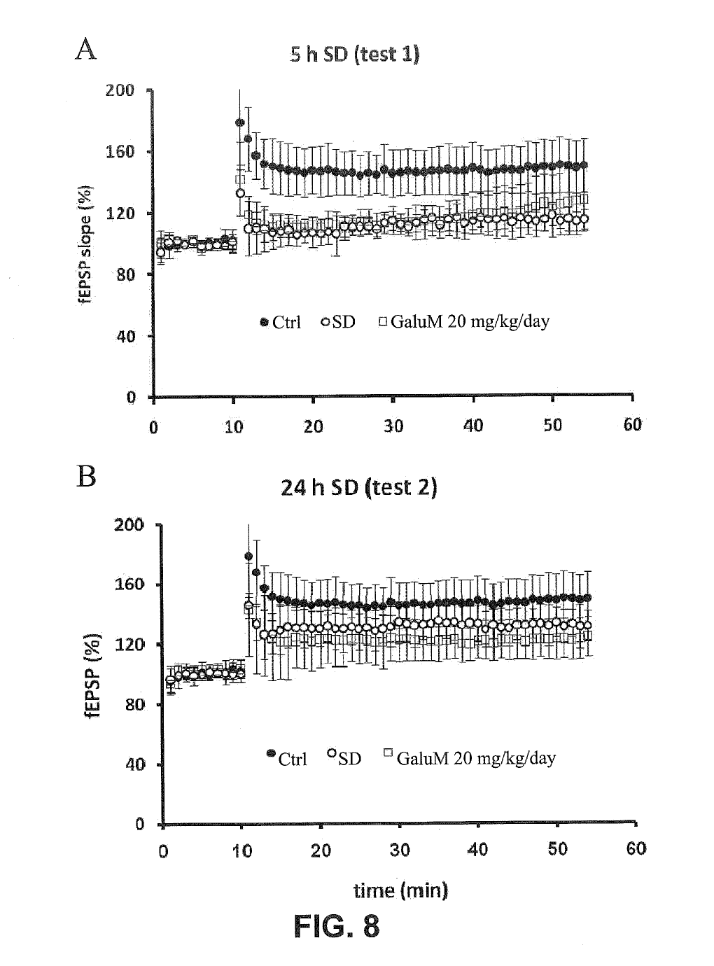

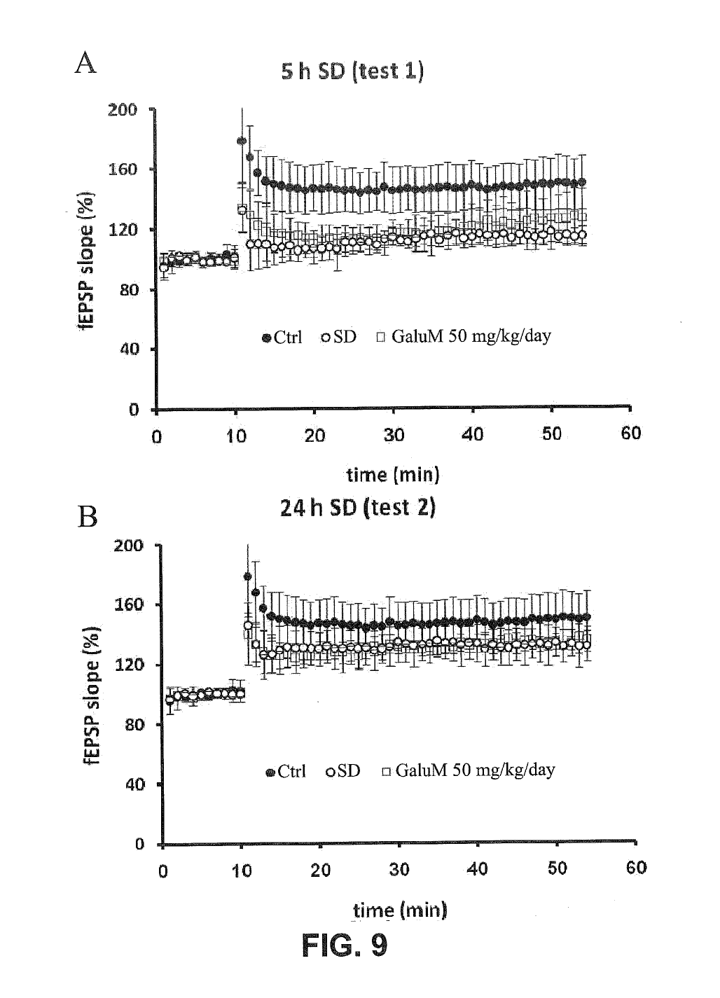

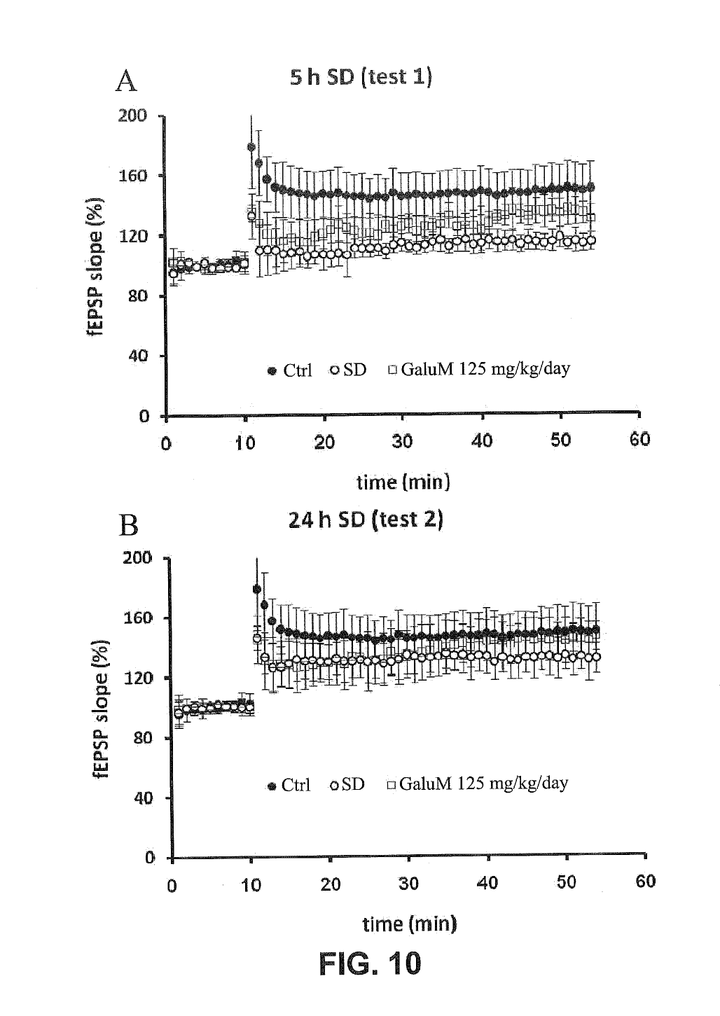

It was observed that sleep deprivation did not severely affect the LTP of mice which were fed Ganoderma lucidum compared to mice which were not fed Ganoderma lucidum. The LTP of mice which were fed Ganoderma lucidum was 133.6±7%, but the LTP of mice which were not fed Ganoderma lucidum was 113.5±5.9%. Over time, the degree of induction of LTP was close to the control group of mice which were never subjected to sleep deprivation (148.5±16.7%) (FIGS. 8A, 9A, 10A). Regarding late-phase long term potential (L-LTP) which may present long tem memory, the group of mice which were fed 125 mg/kg/day Ganoderma lucidum upon sleep depletion demonstrated the result (p=0.001-0.4) close to that of the control group as shown in Table 2. Further, as shown in Table 2, the L-LTP of mice which were fed 20 mg/kg/day and 50 mg/kg/day Ganoderma lucidum were 126.9±22.3% and 127.6±18.6% respectively, and moreover, the LTP of these 2 groups of mice was higher than that of the sleep deprived group of mice. Additionally, after the mice were allowed to sleep freely for 24 hours, the LTPs of mice from the different treatment groups were not significantly different (FIGS. 8B, 9B, 10B). These results showed that under normal physiological conditions, Ganoderma lucidum may not abnormally enhance LTP; however, when other external factors were present, such as sleep deprivation thus affecting memory consolidation, Ganoderma lucidum can prevent or reverse memory disturbances.

TABLE 2
The late phase LTP of mice fed with different doses of GaluM after 5-hour sleep deprivation.

| min | 41 | 42 | 43 | 44 | 45 | 46 | 47 | 48 | 49 | 50 | 51 |
|---|---|---|---|---|---|---|---|---|---|---|---|
| Ctrl | 147.24 ± | 145.23 ± | 146.25 ± | 147.51 ± | 147.14 ± | 146.88 ± | 149.01 ± | 147.98 ± | 149.23 ± | 148.77 ± | 150.27 ± |
| | 15.57 | 14.47 | 14.33 | 14.81 | 15.55 | 15.04 | 16.28 | 16.55 | 17.49 | 16.88 | 17.96 |
| SD | 116.08 ± | 114.79 ± | 114.86 ± | 115.81 ± | 113.12 ± | 115.74 ± | 114.70 ± | 113.65 ± | 114.88 ± | 117.57 ± | 113.58 ± |
| | 6.22^{a} | 7.54^{a} | 6.39^{a} | 6.85^{a} | 5.95^{a} | 6.32^{a} | 5.31^{a} | 5.53^{a} | 7.22^{a} | 6.98^{a} | 4.46^{a} |
| GaluM 20 | 119.03 ± | 118.75 ± | 119.00 ± | 120.31 ± | 118.59 ± | 120.45 ± | 121.61 ± | 123.13 ± | 125.34 ± | 123.19 ± | 124.27 ± |
| mg/kg/day | 13.38^{a} | 14.33^{a} | 16.61^{a} | 16.29^{a} | 16.68^{a} | 17.35^{a} | 16.84^{a} | 19.48^{a} | 18.30 | 20.10^{a} | 19.67^{a} |
| GaluM 50 | 120.75 ± | 124.68 ± | 120.41 ± | 123.47 ± | 122.40 ± | 120.51 ± | 124.28 ± | 126.24 ± | 123.88 ± | 126.41 ± | 125.47 ± |
| mg/kg/day | 12.25^{a} | 13.43^{a} | 14.35^{a} | 16.82^{a} | 14.19^{a} | 12.19^{a} | 19.74^{a} | 21.27 | 14.87^{a} | 21.87 | 15.74^{a} |
| GaluM 125 | 127.08 ± | 130.92 ± | 132.08 ± | 135.71 ± | 132.75 ± | 131.54 ± | 134.78 ± | 134.55 ± | 135.08 ± | 133.47 ± | 136.89 ± |
| mg/kg/day | 7.76^{a} | 4.63^{b} | 6.78^{b} | 24.48^{b} | 6.27^{b} | 5.90^{b} | 8.49^{b} | 10.74^{b} | 7.61^{b} | 9.48^{b} | 11.37^{b} |

^{a}p < 0.05, compare to control group.
^{b}p < 0.05, conpare to SD (sleep deprivation) group.

Ganoderma lucidum Increased Activation of Mice Hippocampal Cell Autophagy and Prevented or Reversed the Memory Loss Due to Sleep Deprivation

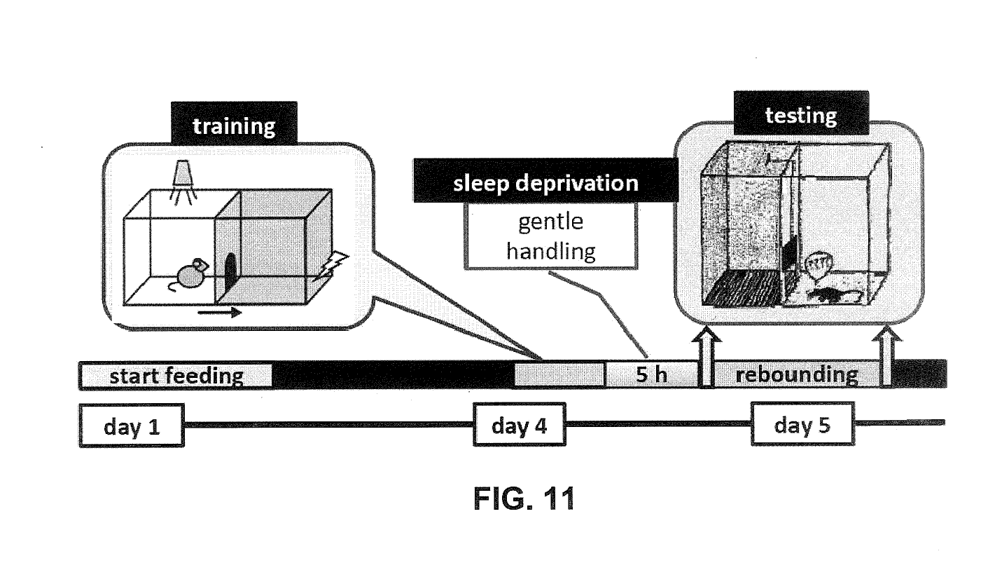

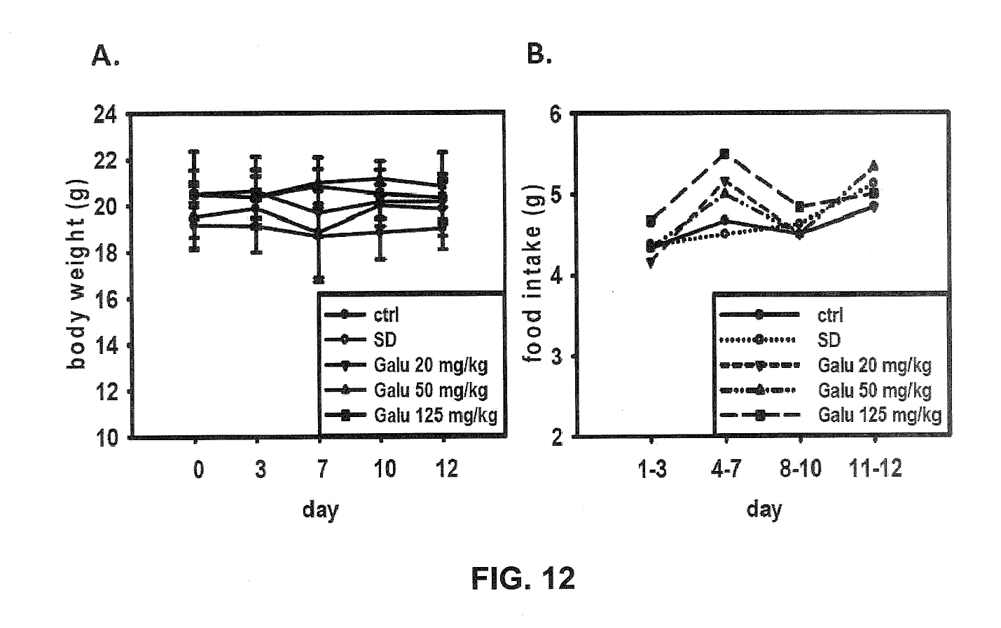

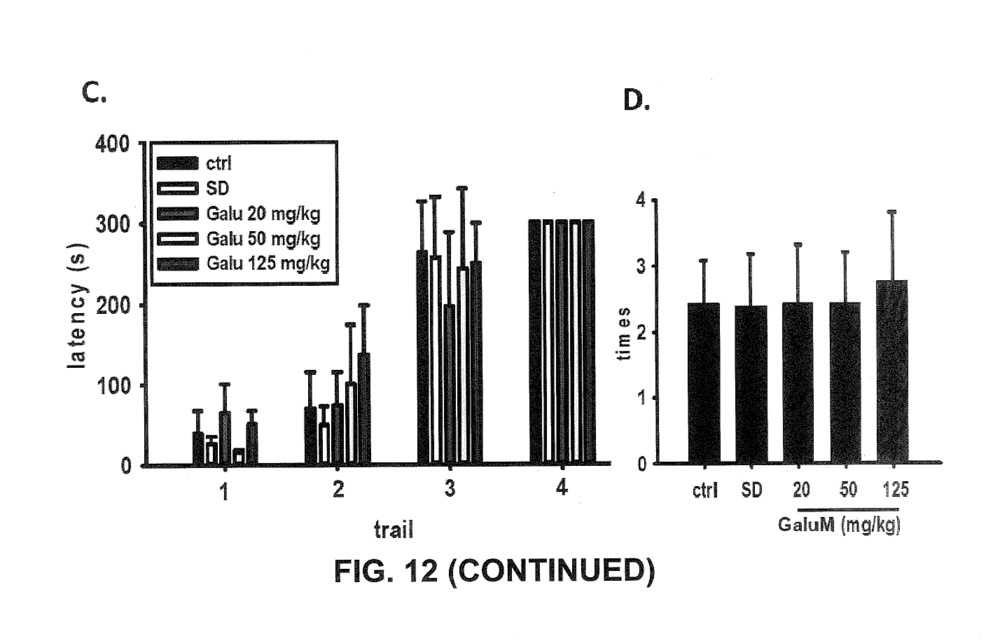

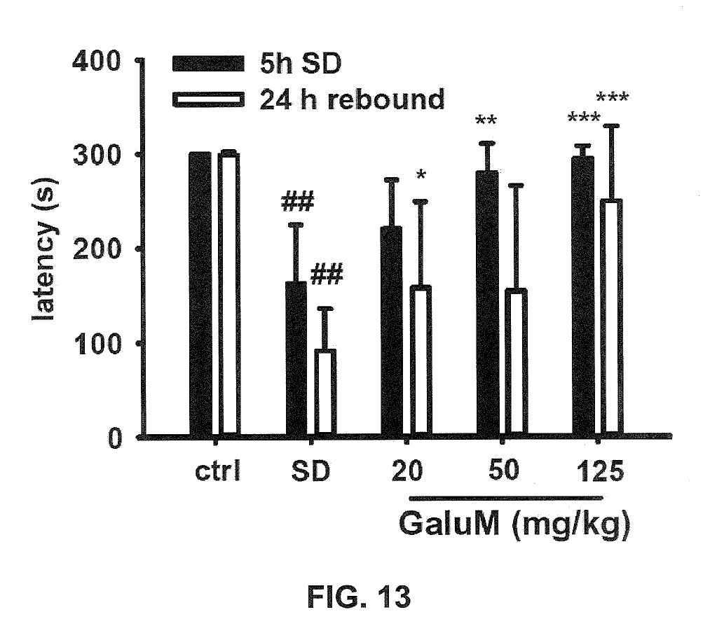

A sleep deprivation model involving the passive avoidance test training was performed on the fourth morning (FIG. 11). The mice were fed Ganoderma lucidum for three days prior to the passive avoidance test training, and this feeding of Ganoderma lucidum did not affect the time that mice required for learning, or the training times of the mice (FIG. 12C). Additionally, the weight gain in mice (FIG. 12A) and manner of feeding (FIG. 12B) were also not affected by the animal feed mixed with Ganoderma lucidum.

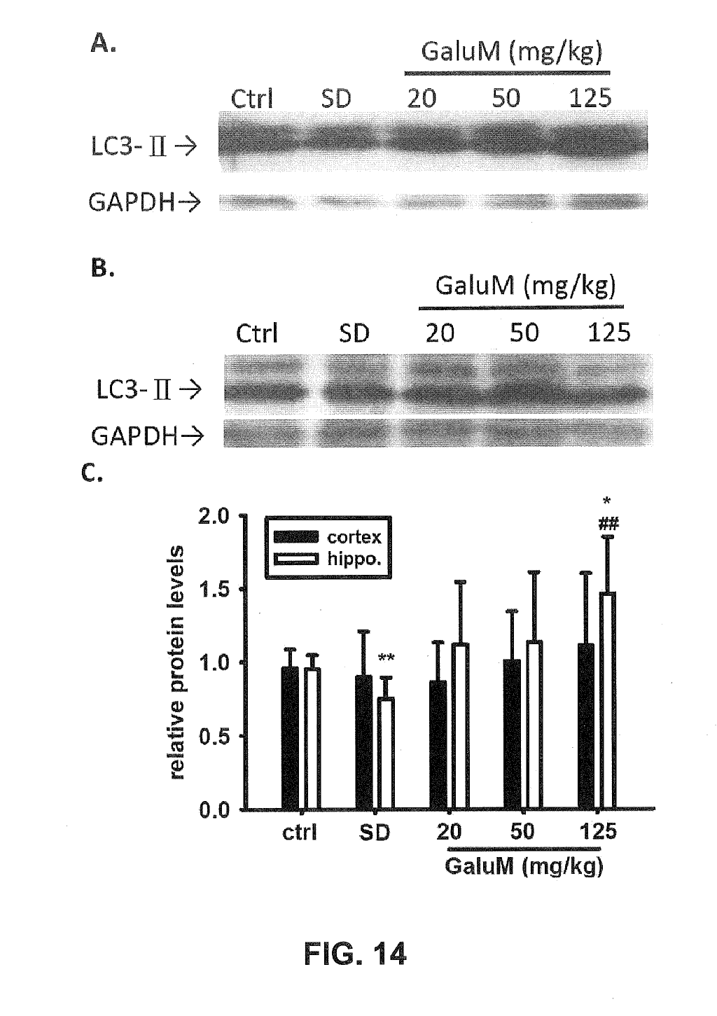

After completion of training, the passive avoidance test was performed upon 5h sleep depletion to test the mice's. It was observed that the control group of mice did not clearly remember their experience of being electroshocked upon entering the dark compartment (latency: 96±72.7) (FIG. 14). However, the Ganoderma lucidum fed group of mice had a latency period of 160.8±20.6˜242.9±89.9, which was worse than the control group but better than the sleep deprived group. These results showed that Ganoderma lucidum may prevent or reverse memory loss resulting from sleep deprivation.

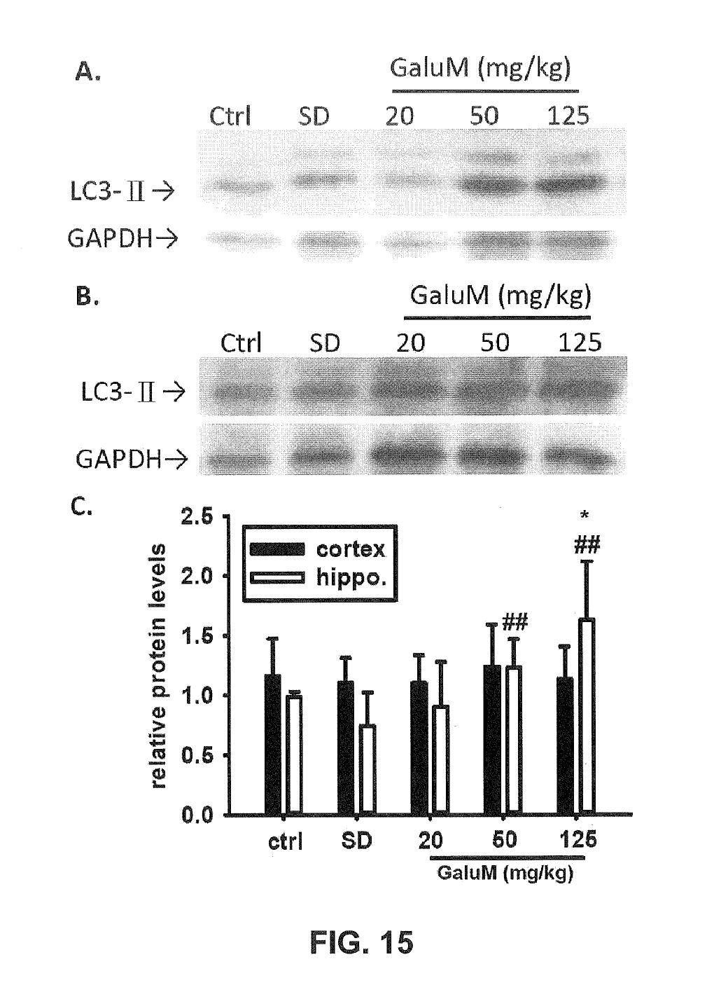

The mice were sacrificed after experimentation, and their hippocampus and cortex were examined for evidence of activation of cell autophagy. Western blot analysis was performed to detect the hippocampus and cortex of the sleep deprived mice (FIG. 14) and the mice that had been fed Ganoderma lucidum for 5 days (FIG. 15). It was shown that the level of cell autophagy was dependent on the dose of Galu fed to the mice (0.9±0.38˜1.63±0.49), and the levels of LC3-II were not statistically significantly different between the different groups of mice cortex. These results indicated that feeding of Ganoderma lucidum activated cell autophagy in the hippocampus in mice. The passive avoidance test showed that the hippocampus is involved in the formation of memory in animal behavior. Therefore, it was observed that the activation of cell autophagy in the hippocampus is involved in the memory consolidation.

The foregoing descriptions are only illustrative of the features and functions of the present invention but are not intended to restrict the scope of the present invention. It is apparent to those skilled in the art that all equivalent modifications and variations made in the foregoing descriptions according to the spirit and principle in the disclosure of the present invention should fall within the scope of the appended claims.

What is claimed is:

1. A method for inducing autophagy in a subject having an autophagy defect, comprising administering to the subject a therapeutically effective amount of a Ganoderma lucidum extract, wherein the autophagy enhances clearance of protein aggregates in the subject.

2. The method of claim 1, wherein the autophagy defect is in a cell expressing the protein aggregates in the subject.

3. The method of claim 2, wherein the cell of the subject is a neuronal cell or glial cell.

4. The method of claim 1, wherein the protein aggregate is an aggregate selected from the group consisting of hungtingtin, amyloid β (Aβ), α-synuclein, tau, superoxide dismutase 1 (SOD 1), variants and mutated forms thereof, and a combination thereof.

5. The method of claim 1, wherein the autophagy defect is one disease selected from the group consisting of neurodegenerative disease, Crohn's disease, aging, heart disease and liver disease.

6. The method of claim 5, wherein the neurodegenerative disease is one selected from the group consisting of Huntington's disease, Alzheimer's disease, Parkinson's disease, amyotrophic lateral sclerosis (ALS) and insomnia.

7. The method of claim 1, wherein the Ganoderma lucidum extract is administered orally to the subject.

8. A method for activating nerve growth factor (NGF) in a subject having an autophagy defect, comprising activating autophagy in the subject by the NGF, wherein the autophagy enhances clearance of protein aggregates in the subject.

9. The method of claim 8, comprising administering to the subject a therapeutically effective amount of a Ganoderma lucidum extract.

10. The method of claim 8, wherein the autophagy defect is in a cell expressing the protein aggregates in the subject.

11. The method of claim 8, wherein the protein aggregate is an aggregate selected from the group consisting of hungtingtin, amyloid β (Aβ), α-synuclein, tau, superoxide dismutase 1 (SOD 1), variants and mutated forms thereof, and a combination thereof.

12. A method for preventing memory loss in a subject, comprising administering to the subject a therapeutically effective amount of a Ganoderma lucidum extract, wherein the Ganoderma lucidum extract activates autophagy in the subject.

13. The method of claim 12, wherein the Ganoderma lucidum extract induces nerve growth factor (NGF) to activate the autophagy in the subject.

14. The method of claim 12, wherein the autophagy enhances protein clearance in the subject.

15. The method of claim 12, wherein the subject has an autophagy defect.

16. The method of claim 15, wherein the autophagy defect is a neurodegenerative disease selected from the group consisting of Huntington's disease, Alzheimer's disease, Parkinson's disease, amyotrophic lateral sclerosis (ALS) and insomnia.

17. A composition for inducing autophagy in a subject having an autophagy defect, comprising one or more of a ganoderic acid and a pharmaceutical acceptable carrier.

18. The composition of claim 17, wherein the ganoderic acid is one or more selected from the group consisting of Ganoderic acid C2, Ganoderic acid A, Ganoderic acid H, Ganoderenic acid D, Ganoderenic acid D, and 12-acetoxyganoderic acid F
